(19) United States
(12) Patent Application Publication
(10) Pub. No.: US 2023/0341404 A1
(43) Pub. Date: Oct. 26, 2023

(54) TARGETING STROMA-CANCER INTERACTIONS IN CANCER

(71) Applicant: The Regents of the University of California, Oakland, CA (US)

(72) Inventors: Michael Karin, La Jolla, CA (US); Hua Su, La Jolla, CA (US)

(21) Appl. No.: 18/138,679

(22) Filed: Apr. 24, 2023

Related U.S. Application Data
(60) Provisional application No. 63/334,486, filed on Apr. 25, 2022.

(51) Int. Cl. G01N 33/574; C12N 15/11; A61P 35/00; C12N 9/22

(52) U.S. Cl. CPC G01N 33/57438; C12N 15/11; A61P 35/00; C12N 9/22; C12N 2310/20; C12N 2800/80

(57) ABSTRACT

Provided herein are prognostic methods for determining median survival of subjects from a cancer such as a desmoplastic cancer, a fibrolytic cancer or a pancreatic ductal adenocarcinoma (PDAC) as well as methods for treating same.

### CROSS-REFERENCE TO RELATED APPLICATION

This application claims priority under 35 U.S.C. § 119(e) to U.S. Provisional Application No. 63/334,486, filed Apr. 25, 2022, the contents of which are incorporated herein by reference in their entireties.

### STATEMENT OF GOVERNMENT SUPPORT

This invention was made with government support under AI043477, CA211794, and DK098108, awarded by the National Institutes of Health (NIH). The government has certain rights in the invention.

### BACKGROUND

Retrospective clinical studies suggest that pancreatic ductal adenocarcinoma (PDAC) patients with a fibrogenic but inert tumor stroma, defined by extensive ECM deposition, low expression of the myofibroblast marker α-SMA and low MMP activity, have improved progression-free survival (PFS) as compared to patients whose tumors are populated by fibrolytic stroma, defined by low collagen fiber content, high α-SMA expression and MMP activity⁹. How the stromal state affects clinical outcome is unknown. Moreover, previous investigations of stromal influence on PDAC growth and progression yielded conflicting results, assigning stroma and CAFs as either tumor supportive^{5,10} or restrictive^{7,8}. It is likely that the failure of stromal-targeted PDAC therapies^{11,12} is due, at least in part, to unrecognized pathways that result in tumor-promoting or tumor-suppressive stromal subgroups, thus requiring precision medicine rather than one-size-fits-all approaches. This disclosure provides diagnostic and therapeutic methods that satisfy this need.

### SUMMARY OF THE DISCLOSURE

Applicant provides herein methods for treating a subject suffering from a cancer selected from a desmoplastic cancer, a fibrolytic cancer or pancreatic ductal adenocarcinoma (PDAC) that has a tumor that has a higher level of cleaved type I collagen (cCol I) as compared to a subject not suffering from the cancer or suffering from the cancer but having a lower level of cCol I and/or comparative better outcome, comprising administering an effective amount of an aggressive anti-tumor therapy or an effective amount of a therapy that inhibits DDR1-stimulated NF-κB or mitochondrial biogenesis. In a further aspect, the tumor further has a higher level of DDR1 and/or NRF2 as compared to a subject not suffering from the cancer or a subject having a lower level of DDR1 and/or NRF2 and suffering from the cancer but having a better comparative outcome. In one aspect, the cancer is pancreatic ductal adenocarcinoma that may be primary or metastatic. In another aspect, the cancer has metastasized to the liver. In another aspect, the level of cCol I is detected by a method comprising immunohistochemical detection of and/or the level of DDR1 and/or NRF2 is detected by a method comprising immunohistochemical detection.

In one embodiment, the treatment comprises one or more of: inhibiting metastatic potential of the cancer; reduction in tumor size; a reduction in tumor burden, longer progression free survival and longer overall survival of the subject.

Also provided are methods for determining if a subject suffering from a cancer selected from a desmoplastic cancer, a fibrolytic cancer or pancreatic ductal adenocarcinoma (PDAC) is more or less likely to experience a longer survival comprising detecting the level of cleaved type I collagen (cCol I), in a tumor sample isolated from the subject, wherein a lower level of cCol I as compared to a subject not suffering from the cancer or as compared to a patient having a higher level of cCol I and having a less favorable outcome, indicates that the subject is more likely to experience longer survival and a higher level of cCol I as compared to a subject not suffering from the cancer indicates that the subject is more likely to experience shorter survival.

### BRIEF DESCRIPTION OF THE DRAWINGS

FIGS. 1A-1G: Col I cleavage controls PDAC growth. FIG. 1A, Immunoblot showing the specificity of antibodies to iCol I and cCol I (3/4 Col I) in ECM produced by the indicated fibroblasts. Col I^{Δ}, Col I knockout; WT, wild type. FIG. 1B, Overall survival of patients with resected PDAC stratified according to cCol I expression (shown in FIG. 5A). Significance was determined by log-rank test. FIG. 1C, Pancreas weight relative to body weight (PB weight) four weeks after orthotopic KPC cell transplantation into Col I^{WT} or Col I^{r/r} mice that were pretreated with CAE or without CAE. Ctrl, control. FIG. 1D, Liver morphology in CAE-treated mice. Liver metastases were detected in 33% of Col I^{WT} mice. FIGS. E, F, Liver gross morphology (FIG. 1E) and tumour numbers (FIG. 1F) two weeks after intrasplenic transplantation of KPC cells into Col I^{WT} or Col I^{r/r} mice with or without CCl₄ pretreatment. FIG. 1G, Representative images and sizes of subcutaneous tumours formed by human 1305 cells co-transplanted with WT, R/R or Col I^{Δ} WT or R/R fibroblasts into Nu/Nu mice. Data in FIG. 1F (n=9 mice), FIG. 1G (n=5 mice) and c are mean±s.e.m. Statistical significance determined by two-tailed t-test. Exact P values in c,f are shown in the Source Data. ****P<0.0001. Scale bars (FIG. 1D, FIG. 1E, FIG. 1G), 1 cm.

FIGS. 2A-2F: Col I cleavage controls PDAC metabolism. FIGS. 2A-2C, Genes differentially expressed between KPC cells grown on wild-type or R/R ECM in LG (0.5 mM) medium for 24 h, lighter gray, replicates with low expression (z-score=−2); darker gray, replicates with high expression (z-score=2). Mitochondrial ETC genes (FIG. 2A), mitochondrial ribosome subunit genes (FIG. 2B) and macropinocytosis-related and NRF2-target genes (FIG. 2C). (FIGS. D, E), Fractional labelling (mole per cent enrichment) of TCA cycle intermediates (FIG. 2D) and intracellular amino acids (FIG. 2E) in KPC cells incubated for 24 h in LG medium after plating on [U-¹³C]-glutamine-labelled wild-type or R/R ECM. α-KG-, α-ketoglutarate. FIG. 2F, KPC cells plated on wild-type or R/R ECM or plastic were incubated in CM or LG medium with or without EIPA, MBQ-167 (MBQ), MRT68921 (MRT), EIPA+MRT or MBQ+MRT for 24 h. Total cellular ATP is presented relative to untreated plastic-plated cells. CM, complete medium. Data in d,e (n=3 per condition) and f (n=3 independent experiments) are mean±s.e.m. Statistical significance determined by two-tailed t-test. Exact P values are shown in the Source Data provided in Su H. et al. (2022) Nature 610, 366-372, incorporated herein by reference. ***P<0.001; ****P<0.0001.

FIGS. 3A-3D: Col I cleavage controls macropinocytosis and the number of mitochondria in PDAC. FIG. 3A, Representative images and rates of macropinocytosis (MP) in TMR-DEX-incubated KPC and MIA PaCa-2 cells grown on plates with or without wild-type or R/R ECM and incubated in LQ or LG medium for 24 h. FIG. 3B, Immunoblot analysis of the indicated proteins in KPC cells treated as in FIG. 3A. FIG. 3C, Representative images of mitochondria (TIM23) in KPC cells grown on plates with or without wild-type or R/R ECM and incubated in LG medium for 24 h. Bottom left, quantification of the number of mitochondria. FIG. 3D, Immunoblot analysis of the indicated proteins in KPC cells treated as in FIG. 3C. Results in FIG. 3A, FIG. 3C (n=6 fields) are mean±s.e.m. Statistical significance determined by two-tailed t-test. ****P<0.0001. Scale bars (FIG. 3A, FIG. 3C), 10 μm.

FIGS. 4A-4G: The Col I—DDR1—NRF2 axis controls macropinocytosis and mitochondrial biogenesis. FIG. 4A, Representative images and quantification of mitochondria and macropinocytosis in TMR-DEX-incubated parental and variant KPC cells grown on wild-type ECM. FIG. 4B, Immunoblot analysis of the indicated proteins in KPC cells grown on plastic or wild-type or R/R ECM and incubated in LG or LQ medium for 24 h. The effects of wild-type and R/R ECM on DDR1 signaling are summarized on the right. mito., mitochondria; pDDR1, phosphorylated DDR1. FIG. 4C, Representative images and quantification of mitochondria and macropinocytosis in TMR-DEX-incubated parental and NRF2^{E79Q} (E79Q) KPC cells plated on wild-type or R/R ECM in LG medium with or without 7rh or ML120B for 24 h. FIG. 4D, Immunoblot analysis of the indicated proteins in parental, E79Q, DDR1^{Δ} and E79Q/DDR1^{Δ} KPC cells plated with or without wild-type or R/R ECM and incubated in LG medium for 24 h. FIG. 4E, Representative IHC of the indicated proteins in Col I^{WT} and Col I^{r/r} pancreata four weeks after KPC cell transplantation. Boxed areas are further magnified. Scale bars, 100 μm. FIG. 4F, Immunoblot analysis of the indicated proteins in KPC cells plated on wild-type or R/R ECM and incubated in LG medium with or without +MG132 or chloroquine (CQ) for 24 h. FIG. 4G, Representative images showing GFP—DDR1 and polyubiquitin (polyub) colocalization in GFP—DDR1-expressing 1305 cells co-cultured with wild-type or R/R fibroblasts in LG medium for 24 h. Boxed areas are further magnified. Data in FIG. 4A, FIG. 4C (n=6 fields) are mean±s.e.m. Statistical significance determined by two-tailed t-test. Exact P values are shown in the Source Data provided in Su H. et al. (2022) Nature 610, 366-372, incorporated herein by reference. ****P<0.0001; NS, not significant. Scale bars (FIG. 4A, FIG. 4C, FIG. 4G), 10 μm.

FIGS. 5A-5B: Col I cleavage and increased DDR1—NRF2 signaling predict poor patient survival. FIG. 5A, Representative IHC of 106 resected human PDACtissues. H&E, haematoxylin and eosin. Boxed areas are further magnified. Scale bars, 100 μm. FIG. 5B, Comparisons of overall survival between patients stratified according to cCol I, DDR1 and NRF2 expression. Significance was determined by log-rank test.

FIGS. 6A-6H: Therapeutic targeting of the DDR1—NF-κB—NRF2 axis inhibits PDAC growth and metabolism. FIG. 6A, Parental and E79Q KPC cells plated on wild-type or R/R ECM were incubated in LG medium with or without 7rh, ML120B or ML385. Total viable cells are presented relative to parental cells that were treated with vehicle and plated on wild-type ECM. FIG. 6B, Oxygen consumption rate (OCR) of parental and E79Q KPC cells plated on wild-type or R/R ECM and incubated in LG medium for 24 h before and after treatment with oligomycin (Omy), FCCP or rotenone/antimycin A. FIG. 6C, Representative images and sizes of parental and NHE1^{KD} MIA tumours grown with or without wild-type or R/R fibroblasts in nude mice. Right, immunoblot analysis of NHE1 in MIA cells. FIGS. D, E, Liver and pancreas morphology (FIG. 6D) and weight (FIG. 6E) four weeks after orthotopic transplantation of KPC E79Q cells into CAE-pretreated Col I^{WT} and Col I^{r/r} mice. FIG. 6F, IHC of pancreatic sections from the mice in FIG. 6D, FIG. 6E. Boxed areas are further magnified. Scale bars, 100 FIG. 6G, PB weight four weeks after orthotopic transplantation of the indicated KPC cells into Col I^{WT} and Col I^{r/r} mice pretreated with or without CAE. Right, immunoblot analysis of DDR1 and Flag-tagged E79Q in the indicated KPC cells plated on wild-type ECM in LG medium for 24 h. FIG. 6H, Representative images and sizes of MIA tumours grown with wild-type or R/R fibroblasts in nude mice with or without ML120B or tigecycline. Data in FIG. 6A (n=3 independent experiments), FIG. 6C, FIG. 6G, FIG. 6H (n=5 mice) and e (n=9 mice) are mean±s.e.m. Statistical significance determined by two-tailed t-test. ***P<0.001, ****P<0.0001; NS, not significant. Exact P values in FIG. 6A, FIG. 6C, FIG. 6G are shown in the Source Data provided in Su H. et al. (2022) Nature 610, 366-372, incorporated herein by reference. Scale bars (FIG. 6C, FIG. 6D, FIG. 6H), 1 cm.

FIGS. 7A-7L Col I cleavage stimulates PDAC growth. FIG. 7A, Overall survival of patients with PDAC from TCGA with high and low collagen-cleaving MMP signature (MMP1, 2, 8, 9, 13, 14). Significance was analyzed by log-rank test. FIG. 7B, UMAPs showing scRNA-seq data from 5 primary PDACs, displaying cell types and expression of the most abundant WP mRNAs. FIG. 7C, Pancreas morphology 4 wk after orthotopic KPC cell transplantation into Col I^{WT} or Col I^{r/r} mice−/+CAE pretreatment. FIG. 7D, H&E and sirius red (SR) staining of pancreatic sections from above mice. Boxed areas were further magnified. Quantification of SR positivity in nontumor (NT) areas is shown to the right. FIG. 7E, IHC of pancreatic sections from above mice. Quantification of tumour areas is shown to the right. FIG. 7F, H&E, SR, Ki67 staining of liver sections from above CAE-pretreated mice. FIG. 7G, Liver gross morphology and tumour numbers (#) 2 wk after i.s. transplantation of Paren. or 1KKα knockdown (KD) KC cells into CCl₄ pretreated Col I^{WT} or Col I^{r/r} mice. FIG. 7H, H&E and SR staining of liver sections 2 wk after i.s. transplantation of KPC cells into Col I^{WT} and Col I^{r/r} mice−/+CCl₄ pretreatment. Quantification of SR positivity in NT areas is shown at the bottom. FIG. 7I, IHC of liver sections from above mice. Boxed areas show higher magnification. Results in (FIG. 7E) (n=5 fields), (FIG. 7G), (h) (n=6 mice) and (FIG. 7D) are mean±s.e.m. Statistical significance determined by two-tailed t-test. ***P<0.001, ****P<0.0001. Scale bars in (FIGS. 7D-7F, and FIG. 7H, FIG. 7I), 100 μm, (FIG. 7C, FIG. 7G), 1 cm.

FIGS. 8A-8J: The Col I state controls PDAC gene expression and metabolism. FIG. 8A, Dataset enrichment of RNA-seq data (n=3) from KPC cells plated on wild-type (WT) (light gray) or R/R (light gray) ECM and incubated in LG for 24 h. FIG. 8B, KPC cells grown on [³H]-proline-labelled WT, R/R, Col I^{Δ} R/R or Col I^{Δ} R/R +Col I^{WT} ECM were incubated in LG−/+EIPA for 24 h. [³H] uptake is presented relative to vehicle treated WT ECM-plated KPC cells. IB analysis of iCol I and 3/4 Col I in ECM produced by indicated fibroblasts. FIG. 8C, Indicated KPC cells were plated on [³H]-proline-labelled ECM and incubated in LG for 24 h. [³H] uptake is presented relative to Paren. uptake. KD efficiency is demonstrated. FIG. 8D, KPC cells were plated on [³H]-proline-labelled ECM and incubated in LG −/+ indicated reagents for 24 h. [³H] uptake is presented as above. FIG. 8E, [³H] uptake by KC cells treated as above. FIG. 8F, AA content of ECM-plated KPC cells incubated in LG−/+indicated reagents for 24 h. Cell number normalized data are presented relative to untreated WT ECM-plated cells. FIG. 8G, KC cells were plated −/+WT or R/R ECM and incubated in complete (CM) or LG media −/+ indicated reagents for 24 h. Cellular ATP content is presented relative to untreated plastic-plated cells. FIG. 8H, KPC cells were plated as in (FIG. 8B) and incubated in LG −/+ EIPA for 24 h. Cellular ATP content is presented relative to untreated WT ECM-plated cells. FIG. 8I, Total AA in KC cells plated on ECM and incubated in LG −/+ indicated reagents for 24 h. Data are presented as above. FIG. 8J, Total AA in KPC cells treated as in (FIG. 8H). Results in (FIG. 8B, FIGS. 8D-8J) (n=3 independent experiments), (FIG. 8C) (n=4 independent experiments) are mean±s.e.m. Statistical significance determined by two-tailed t-test. **P<0.01, ***P<0.001, ****P<0.0001. Exact P values in (FIGS. 8B-8J) are shown in Source Data provided in Su H. et al. (2022) Nature 610, 366-372, incorporated herein by reference.

FIGS. 9A-9G: The cleaved to intact Col I ratio controls macropinocytosis and mitochondrial biogenesis. FIG. 9A, Macropinocytosis (MP) visualization and quantification using TMR-DEX in KPC cells co-cultured with WT or R/R fibroblasts and incubated in LG medium for 24 h. KPC cells were marked by cytokeratin 18 (CK18, gray). FIG. 9B, Representative images, and MP quantification in TMR-DEX-injected pancreatic tissue from Col I^{WT} or Col I^{r/r} mice 4 wk after orthotopic KPC cell transplantation. Carcinoma cells are marked by EpCAM staining (gray). Quantification is on the right. FIG. 9C, qRT-PCR analysis of MP-related mRNAs in liver tumours 2 wk after i.s. KPC cell transplantation into CCl₄ pretreated Col^{WT} or Col^{r/r} mice. Exact P values are shown in Source Data. FIG. 9D, IB analysis of MP-related proteins in above liver tumours. FIG. 9E, Representative images, and quantification of Mito. (ATP5B, gray) in pancreatic tissue from indicated mice analysed as in (FIG. 9B). Carcinoma cells are marked by EpCAM staining (dark gray). Quantification is on the right. FIG. 9F, FIG. 9G, Representative images, and quantification of Mito. (TIM23) and MP in TMR-DEX-incubated KPC cells grown on mixed ECM produced by R/R and WT (R:W) (FIG. 9F) or R/R and Col I^{Δ} R/R (R:KO) (FIG. 9G) fibroblasts in the indicated ratios and incubated in LG medium for 24 h. IB analysis of iCol I or cCol I (3/4 Col I) in above ECM preparations is shown at the bottom. Results in (FIG. 9A, FIG. 9F, FIG. 9G) (n=6 fields), (FIG. 9B, FIG. 9C, FIG. 9E) (n=4 mice) are mean±s.e.m. Statistical significance determined by two-tailed t-test. *P<0.05, **P<0.01, ***P<0.001, ****P<0.0001. Scale bars, 10 μm.

FIGS. 10A-10G: Col I controls macropinocytosis and mitochondrial content through DDR1—NRF2 signaling. FIG. 10A, TB analysis of KPC cells ablated for indicated collagen receptors. FIG. 10B, UMAPs showing scRNA-seq data from 5 primary PDAC (upper row) and 1 PDAC liver metastasis (lower row), displaying the identified cell populations and expression of the indicated mRNAs. FIG. 10C, Representative images, and quantification of Mito. and MP in TMR-DEX- incubated Paren. and NRF2^{Δ} (KO) KPC cells plated on WT or R/R ECM and incubated in LG for 24 h. NRF2 IB analysis is shown on the right. FIG. 10D, MP and NRF2 localization in Paren. and NRF2^{KD} MIA cells plated −/+WT or R/R ECM and incubated in LG for 24 h. MP quantification is shown on the right. FIG. 10E, 3/4 Col I and MP imaging and quantification in KPC cells treated as above. Although NRF2^{E79Q} (E79Q) stimulates MP, cCol I uptake is detected only in cells plated on WT ECM. FIG. 10F, Representative images, and quantification of MP and nuclear NRF2 in 1305 cells plated on WT ECM and incubated in LG for 24 h. IB analysis of DDR1 and Flag-tagged E79Q is shown on the right. FIG. 10G, MP imaging and quantification in Paren. and E79Q MIA cells plated on WT or R/R ECM and incubated in LG for 24 h. Results in (FIGS. 10C-10G) (n=6 fields) are mean±s.e.m. Statistical significance was determined by two-tailed t-test. ****P<0.0001. Scale bars, 10 μm.

FIGS. 11A-11G: cCol I—DDR1—NRF2 signaling controls macropinocytosis and mitochondrial protein expression. FIG. 11A, IB analysis of ETC complexes I-V (CI-CV) in Paren. and E79Q KPC cells plated on WT or R/R ECM and incubated in LG for 24 h. FIG. 11B, IB analysis of indicated KPC cells −/+ectopic p62 or E79Q plated on WT ECM and incubated in LG for 24 h. FIG. 11C, IB of ETC proteins in Paren., DDR1^{Δ}, or E79Q/DDR1^{Δ} KPC cells plated on WT ECM and incubated in LG for 24 h. FIG. 11D, FIG. 11E, IB of indicated proteins in Paren. or E79Q KPC cells plated on WT ECM and incubated in LG medium −/+ML120B for 24 h. FIG. 11F, Staining intensity of the indicated proteins in tumour areas depicted FIG. 4E determined with Image J. FIG. 11G, IHC of liver sections prepared 2 wk after i.s. transplantation of KPC cells into CCl₄ pretreated Col I^{WT} and Col I^{r/r} mice. Scale bars, 100 Image J determined staining intensity of indicated proteins in tumour areas is shown at the bottom. Results in (FIG. 11F, FIG. 11G) (n=6 fields) are mean±s.e.m. Statistical significance determined by two-tailed t-test. ****P<0.0001.

FIGS. 12A-12M: Inhibition of Col I cleavage shuts down NRF2-driven macropinocytosis and mitochondrial biogenesis. FIG. 12A, MP and mitochondria in KPC cells plated on indicated ECM and incubated in LG for 24 h. Col IIB in indicated ECM preparations is shown on the right. FIG. 12B, IB of indicated proteins in above cells. FIG. 12C, MP and mitochondria in indicated KPC cells plated on R/R Col I^{Δ} ECM and incubated in LG for 24 h. FIG. 12D, LB of indicated proteins in 1305 cells plated on ECM produced by bacterial collagenase (CLG, 50 μg/ml) treated or untreated fibroblasts and incubated in LG medium −/+CLG for 24 h. FIG. 12E, Genes differentially expressed between KPC cells plated on indicated ECM. Light gray: replicates with low expression (z-score=−2); dark gray: replicates with high expression (z-score=+2). FIG. 12F, Immunoprecipitation (IP) of GFP—DDR1 from 1305 cells plated −/+ indicated ECM. FIG. 12G, MP and mitochondria in Paren. or E79Q KPC cells plated on ECM produced by Ilomastat (Iloma.) treated or untreated WTfibroblasts and incubated in LG medium −/+ Iloma. for 24 h. FIG. 12H, IB of indicated proteins in above cells. FIG. 12I, MP and mitochondria in ATG7^{Δ} MIA PaCa-2 cells plated on indicated ECM and incubated in LG for 24 h. FIG. 12J, Imaging of 1305 cells plated on indicated ECM showing rare poly-Ub and Mito. (Tom20) colocalization. FIG. 12K, IB analysis of KPC cells plated −/+ indicated ECM and incubated in indicated media for 24 h. FIG. 12L, Locations of putative NRF2-binding sites (AREs) relative to the transcriptional start site (TSS, +1) of the mouse Tfam gene. FIG. 12M, Chromatin IP probing NRF2 recruitment to the Tfam promoter in KPC cells plated on WT or R/R ECM and incubated in LG for 24 h. The image shows PCR-amplified ARE-containing promoter DNA fragments. Quantitation on the right. Results in (FIG. 12A, FIG. 12C, FIG. 12G, FIG. 12I) (n=6 fields), (FIG. 12M) (n=3 independent experiments) are mean±s.e.m. Statistical significance determined by two-tailed t-test. ***P<0.001, ****P<0.0001. Scale bars, 10 μm.

FIGS. 13A-13C: Correlation between Col I—DDR1—NRF2 signaling components and inflammation in human PDAC. FIG. 13A, Numbers and percentages of human PDAC specimens (n=106 specimens) positive for the indicated proteins (arbitrarily indicated as low and high). FIG. 13B, Correlation between indicated proteins in above specimens was analysed by a two-tailed Chi-square test. *P<0.05, **P<0.01, ***P<0.001, ****P<0.0001. cCol I (3/4 Col I). FIG. 13C, Representative IHC and quantification of the indicated markers in cCol I^{high} (#43) and cCol I^{low} (#54) human PDAC specimens from FIG. 5A. Mean ±s.e.m. (n=16 specimens). Statistical significance determined by two-tailed t-test. ***P=0.0001. *P=0.0145, *P=0.0388. Scale bars, 100 μm.

FIGS. 14A-14J: Effect of macropinocytosis and mitochondria on Col I-controlled PDAC cell growth. FIG. 14A, Bromodeoxyuridine (BrdU) incorporation into KPC cells plated on ECM mixtures produced by R/R and WT fibroblasts (R:W). Scale bars, 10 μm. FIG. 14B, FIG. 14C, Paren. and IKKα^{KD} KPC (FIG. 14B) or KC (FIG. 14C) cells were plated −/+ WT or R/R ECM and incubated in LG −/+ indicated reagents. Viable cells were measured after 3 days and depicted relative to untreated plastic-plated Paren. cells. (FIG. 14C). IKKα KD efficiency is shown on the right (FIG. 14B). FIGS. D, E, Viable 1305 (FIG. 14D) or MIA PaCa-2 (FIG. 14E) cells plated as above and incubated in LG −/+ EIPA. f, Viable Paren. and E79Q KPC cells plated, treated, and presented as above. FIG. 14G, Paren. and DDRI^{KD} 1305 or KPC cells plated on indicated ECM preparations and incubated in LG medium −/+ indicated tigecycline concentrations for 24 h. Total viable cells were measured as above and are presented relative to the untreated cells. FIG. 14H, Mitochondrial ATP production calculated from FIG. 6B. FIG. 14I, Liver morphology and tumour numbers (#) 2 wk after i.s. transplantation of Paren. or NHE1^{KD} KPC cells into CCl₄-pretreated Col I^{WT} and Col I^{r/r} mice−/+EIPA. NHE1 IB is shown on bottom left. ****P<0.0001. FIG. 14J, H&E and SR staining of s.c. tumours from FIG. 6C. Quantification of the SR positive area is shown on the left. Scale bars, 100 μm. Results in (FIG. 14A) (n=6 fields), (FIGS. 14B-14G) (n=3 independent experiments), (FIG. 14H) (n=3 per condition), (FIG. 14J) (n=5 mice) and (FIG. 14I) are mean±s.e.m. Statistical significance determined by two-tailed t-test. Exact P values in (FIGS. 14A-14C, FIG. 14F, FIG. 14H) are shown in Source Data provided in Su H. et al. (2022) Nature 610, 366-372, incorporated herein by reference.

FIGS. 15A-15E: The cCol I—DDR1—NRF2 axis controls PDAC growth but not collagen fiber alignment. FIG. 15A, H&E, SR and cytokeratin 19 (CK19) and SOX9 staining of pancreatic and liver sections from CAE-pretreated Col I^{WT} and Col I^{r/r} mice 4 wk after orthotopic KPC NRF2^{E79Q} cell transplantation. Scale bars, 100 μm. FIG. 15B, Tumours formed by orthotopically transplanted Paren. or DDR1^{KD} KPC cells in Col I^{WT} and Col I^{r/r} mice analysed by SHG and collagen fiber individualization. BF-bright field. FIG. 15C, IHC of indicated markers in pancreatic sections of Col I^{WT} and Col I^{r/r} mice orthotopically transplanted with KPC cells. Quantification of staining positivity in tumour areas is shown below. Left to right: ****P<0.0001, **P=0.0013, P=0.5351. FIG. 15D, Pancreas morphology 4 wk after orthotopic transplantation of Paren., DDR1^{KD}, E79Q/DDR1^{KD} KPC cells into Col I^{WT} and Col I^{r/r} mice pretreated −/+CAE. Scale bars, 1 cm. FIG. 15E, H&E staining and IHC analysis of pancreatic sections from above mice. Quantification of tumour area by Image J is shown below. Left to right: ****P<0.0001, P=0.5748, P=0.3606. Results in (FIG. 15C, FIG. 15E) (n=5 fields) are mean±s.e.m. Statistical significance determined by two-tailed t-test. Scale bars, 100 μm.

FIGS. 16A-16D: cCol I stimulates PDAC growth, macropinocytosis and mitochondrial biogenesis through the DDR1—NRF2 axis. FIG. 16A, IHC of pancreatic sections of CAE-pretreated Col I^{WT} or Col I^{r/r} mice 4 wk after orthotopic transplantation of Paren., DDR1^{KD} and E79Q/DDR1^{KD} KPC cells. Boxed areas were further magnified. Scale bars, 100 μm. FIG. 16B, Representative images and sizes of s.c. tumours generated by Paren., DDR1^{KD} (KD) and E79Q/ KD 1305 cells transplanted −/+ WT or R/R fibroblasts into Nu/Nu mice. Scale bars, 1 cm. FIG. 16C, Pancreas weight relative to body weight (PB) of Col I^{WT} or Col I^{r/r} mice 4 wk after orthotopic transplantation of Paren., NRF2^{KD} and TFAM^{KD} KPC cells. NRF2 and TFAM KD efficiency is shown below. FIG. 16D, H&E staining and CK19 IHC of above pancreata. Image J quantification of tumour area is shown on the left. Scale bars, 100 μm. Results in (FIG. 16B, FIG. 16C) (n=5 mice), (FIG. 16D) (n=5 fields) are mean±s.e.m. Statistical significance was determined by a two-tailed t-test. ****P<0.0001.

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure several technical references are indicated by the first author's name and year of publication. The full bibliographic citations for these publications are found immediately preceding the claims.

As used in the specification and claims, the singular form “a”, “an” and “the” include plural references unless the context clearly dictates otherwise. For example, the term “a polypeptide” includes a plurality of polypeptides, including mixtures thereof.

As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, the term “animal” refers to living multi-cellular vertebrate organisms, a category that includes, for example, mammals and birds. The term “mammal” includes both human and non-human mammals. As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis or prevention.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “composition” is intended to mean a combination of active agent and another compound or composition, inert (for example, a detectable agent or label) or active, such as an adjuvant , diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, a purification label or maker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

As used herein, the term “sample” and “biological sample” and “agricultural sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, blood, serum, mucus, bone marrow, lymph, and tears. In some aspects, agricultural samples include soil, foliage or any plant tissue or surface or other sample suspected of harboring virus. In addition, the sample can include industrial samples, such as those isolated from surfaces and the environment.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

“Host cell” refers not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. The host cell can be a prokaryotic or a eukaryotic cell.

In some embodiments, the cell or host cell as disclosed herein is a eukaryotic cell or a prokaryotic cell.

“Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “a regulatory sequence”, “an expression control element”, or “promoter” as used herein, intends a polynucleotide that is operatively linked to a target polynucleotide to be transcribed and/or replicated, and facilitates the expression and/or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters.

A polymerase II or “pol II” promoter catalyzes the transcription of DNA to synthesize precursors of mRNA, and most shRNA and microRNA. Examples of pol II promoters are known in the art and include without limitation, the phosphoglycerate kinase (“PGK”) promoter; EF1-alpha; CMV (minimal cytomegalovirus promoter); and LTRs from retroviral and lentiviral vectors.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

“Administration” can be effected in one dose, continuously or intermittently throughout the course of treatment. Methods of determining the most effective means and dosage of administration are known to those of skill in the art and will vary with the composition used for therapy, the purpose of the therapy, the disease being treated, the target cell being treated, and the subject being treated. Single or multiple administrations can be carried out with the dose level and pattern being selected by the treating physician or treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, inhalation administration, nasal administration, intravenous administration, injection, and topical application.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“An effective amount” or a “therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

“Systemic” or “systemic administration” refers to a route of administration of medication or other substance into the circulatory system such that the entire body may effected. Systemic administration may occur via, for example, intravenous, subcutaneously, topical, oral, or pulmonary administration

“Local” or “local administration” refers to the administration of a medication or other substance at the site of where the desired treatment is required. For example, a medication may be delivered directly to the site of disease (i.e.: the pancreas, liver, lungs, respiratory system, or pulmonary system). Local administration may be accomplished by infusion, injection, inhalation, through the pulmonary system, via injection to the target area, or any other route capable of direct administration to an area where the treatment is desired.

As used herein, “cancer” or “malignancy” or “tumor” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

A desmoplastic cancer is a soft tissue cancer or sarcoma. One type of desmoplastic cancer is a small round cell tumor (DSRCT) that often begins in the abdomen but may occur in other parts of the body. Desmoplastic small round cell tumors are rare cancers. Conventional treatment includes surgery, chemotherapy and radiation therapy. Aggressive treatments include multimodal therapies (e.g., a P6 protocol, which had seven courses of chemotherapy. Courses 1, 2, 3, and 6 included cyclophosphamide 4,200 mg/m2, doxorubicin 75 mg/m2, and vincristine (HD-CAV). Courses 4, 5, and 7 consisted of ifosfamide 9 g/m2 and etoposide 500 mg/m2 for previously untreated patients, or ifosfamide 12 g/m2 and etoposide 1,000 mg/m2 for previously treated patients. Courses started after neutrophil counts reached 500/microL and platelet counts reached 100,000/microL. Tumor resection can be attempted. Post-P6 treatment options included radiotherapy and a myeloablative regimen of thiotepa (900 mg/m2) plus carboplatin (1,500 mg/m2), with stem-cell rescue. Kushner et al. (1996) J. Clin. Oncol. 14:5:1526-1531. Another exemplary aggressive therapy includes administration of immunotherapy or radioimmunotherapy following debulking surgery. Espinosa-Cotton and Dheung (2021), Front. Oncol. 11:772862.10.3389/fonc.2021.772862. See also Bulbul et al. (2017) Desmoplastic Small Round Blue Cell Tumor: A Review of Treatment and Potential Therapeutic Genomic Alterations, Sarcoma, Article ID 1278268.

100701 Pancreatic ductal adenocarcinoma (PDAC) is one of the major pancreatic exocrine cancer with a poor prognosis and growing prevalence. It has an overall five-year survival rate of 6% to 10%. It has been reported that pancreatic cancer stem cells (PCSCs) are the main factor responsible for the tumor development, proliferation, resistance to anti-cancer drugs, and. recurrence of tumors after surgery. Conventional therapies include treatment with cytotoxic agents: FOLFIRINOX (a mixture of Leucovorin and other chemotherapy medicines: Fluorouracil (5FU), Irinotecan and Oxaliplatin]) or Gemcitabine/Nab-paclitaxel. An alternative aggressive therapy includes administration of stem cells such as mesenchymal stem cells. See Thakur, et al. (2021) Biomedicines February. 11;9(2):178. Additional aggressive therapies include multimodal therapies including the administration of two or chemotherapies with or without radiation or tumor resection, personalized therapies and neoadjuvant therapies (see Hamad et al. World J. Gastroenterol. July 21; 27:4383-4394) or triple immunotherapy (Gulhati et al. (2022) Nature Cancer DOI: 10.1038/s43018-022-00500-z. Additional aggressive combination therapies are disclosed in Hosein et al. (2022) Nature Cancer 3:272-286 and include treatments that target DDR1—IKKβ—NF-κB—NRF2 signaling and mitochondrial biogenesis that include stromal state—an important modifier of tumour growth—as an integral biomarker. Given that three Col I-cleaving MMPs were highly expressed in the human PDAC samples Applicant specific MMP inhibitors are additional candidates for aggressive or precision therapy.

These aggressive therapies can be administered for the treatment of fibrolytic cancer as well. A fibrolytic cancer is one having fibrolytic stroma, defined by low collagen fiber content, high α-SMA expression and MMP activity.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

### Modes for Carrying Out the Disclosure

### Prognostic Methods

This disclosure provides methods for determining if a subject suffering from a cancer selected from a desmoplastic cancer, a fibrolytic cancer or pancreatic ductal adenocarcinoma (PDAC) is more or less likely to experience a longer survival. The method comprises, or consists essentially of, or yet further consists of detecting the level of cleaved type I collagen (cCol I), in a tumor sample isolated from the subject, wherein a higher level of cCol I as compared to the cCol I level a subject not suffering from the cancer indicates that the subject is less likely to experience longer survival and a lower level of cCol I as compared to the cCol I level in a subject not suffering from the cancer indicates that the subject is less likely to experience shorter survival. Stated another way, subjects with lower levels of cCol I are more likely to experience a longer survival and subjects with higher levels of cCol I are less likely to experience a longer survival. Survival can be determined by overall survival or progression free survival.

In a further aspect, the methods further comprise, or consist essentially of, or yet further consist of detecting the level of DDR1 and/or NRF2 in the sample, wherein a higher level of DDR1 and/or NRF2 in the sample indicates that the subject is less likely to experience longer survival and subjects with lower levels of DDR1 and/or NRF2 are more likely to have longer survival. Survival can be determined by overall survival or progression free survival.

In one aspect, wherein the cancer is pancreatic ductal adenocarcinoma.

In another aspect, the tumor sample can be a needle biopsy or obtained from tumor resection.

For the purpose of these methods, wherein the cancer is primary or metastatic. In a further aspect, the cancer has metastasized to the liver.

Any known method can be used to determine the level of cCol I. Non-limiting examples include PCR or immunohistochemical detection using anti-cCol I antibodies that optionally can be detectably labeled. Similarly, any method can be used to determine the level of DDR1 and/or NRF2. Non-limiting examples include PCR or immunohistochemical detection using anti-DDR1 or anti-NRF2 antibodies that optionally can be detectably labeled.

The methods can further comprise, or consist essentially of, or yet further consist of administering to the subject having a higher level of any one, two or three of cCol I, DDR1, and NRF2, an aggressive anti-tumor therapy, such immune therapy or chemotherapy including multiple rounds of each. Alternatively, the methods further comprise, or consist essentially of, or yet further consist of, administering to the subject having a higher level of any one, two or three of cCol I, DDR1, and NRF2, an effective amount of a therapy that inhibits DDR1-stimulated NF-κB or mitochondrial biogenesis.

The above methods for determining the levels of one or more of cCol I, DDR1, and/or NRF2 can be repeated during the course of such therapy.

The methods can be practiced on subjects that are mammals, e.g., a canine, feline, equine, murine, or a human patient.

In a further aspect, the method further comprises isolating a tumor sample, by tumor resection, liquid biopsy or needle biopsy.

### Therapeutic Methods

Also provided herein are method for treating a subject suffering from a cancer selected from a desmoplastic cancer, a fibrolytic cancer or pancreatic ductal adenocarcinoma (PDAC) that has a tumor that has a higher level of cleaved type I collagen (cCol I) as compared to the level of cCol I in a subject not suffering from the cancer, comprising, or consisting essentially of or yet further consisting of administering an effective amount of an aggressive anti-tumor therapy or an effective amount of a therapy that inhibits DDR1-stimulated NF-κB or mitochondrial biogenesis. In one aspect, the cancer is PDAC. Non-limiting examples of aggressive therapy include immune therapy or chemotherapy including multiple rounds of each.

Administration can be accomplished by the method and dosage as determined by the treating physician or as known to those of skill in the art. Exemplary aggressive therapies are known in the art and described herein for PDAC, fibrolytic and desmoplastic cancer, several of which are identified herein.

In another aspect, the tumor is determined to a higher level of DDR1 and/or NRF2 as compared to a subject not suffering from the cancer and/or a level from a cancer patient that exhibited a more favorable outcome.

In another aspect, the cancer is PDAC and the cancer is primary or metastatic. In a further aspect, the cancer has metastasized to the liver. The treatment can be first-line, second-line, third-line, or fourth line therapy.

Any known method can be used to determine the level of cCol I. Non-limiting examples include PCR or immunohistochemical detection using anti-cCol I antibodies that optionally can be detectably labeled. Similarly, any method can be used to determine the level of DDR1 and/or NRF2. Non-limiting examples include PCR or immunohistochemical detection using anti-DDR1 or anti-NRF2 antibodies that optionally can be detectably labeled.

The methods can be practiced on subjects that are mammals, e.g., a canine, feline, equine, murine, or a human patient. The treatment can be a first line, second line, third line, fourth line or fifth line therapy. The methods can be combined with other known therapies, such as tumor resection. Moreover, the levels of cCol I, DDR1 and/or NRF2 can be detected from a tumor sample isolated by any known method, e.g., comprising a liquid biopsy, tumor resection or tumor biopsy.

One of skill of art will know when the method has been successful, non-limiting clinical endpoints include one or more of: inhibiting metastatic potential of the cancer; reduction in tumor size; a reduction in tumor burden, longer progression free survival and longer overall survival of the subject.

The subject to be treated can be an animal or a human. When practiced on an animal such as a mouse or other mammal, the treatment can be used for testing other therapies or new combination therapies. When administered to a human, it can be used for a more favorable outcome for the patient, such as longer overall or progression free survival.

### Experimental

Pancreatic ductal adenocarcinoma (PDAC) is a highly desmoplastic, aggressive cancer that frequently progresses and spreads by metastasis to the liver¹. Cancer-associated fibroblasts, the extracellular matrix and type I collagen (Col I) support^{2,3} or restrain the progression of PDAC and may impede blood supply and nutrient availability⁴. The dichotomous role of the stroma in PDAC, and the mechanisms through which it influences patient survival and enables desmoplastic cancers to escape nutrient limitation, remain poorly understood. Here Applicant shows that matrix-metalloprotease-cleaved Col I (cCol I) and intact Col I (iCol I) exert opposing effects on PDAC bioenergetics, macropinocytosis, tumour growth and metastasis. Whereas cCol I activates discoidin domain receptor 1 (DDR1)—NF-κB—p62—NRF2 signaling to promote the growth of PDAC, iCol I triggers the degradation of DDR1 and restrains the growth of PDAC. Patients whose tumours are enriched for iCol I and express low levels of DDR1 and NRF2 have improved median survival compared to those whose tumours have high levels of cCol I, DDR1 and NRF2. Inhibition of the DDR1-stimulated expression of NF-κB or mitochondrial biogenesis blocks tumorigenesis in wild-type mice, but not in mice that express MMP-resistant Col I. The diverse effects of the tumour stroma on the growth and metastasis of PDAC and on the survival of patients are mediated through the Col I—DDR1—NF-κB—NRF2 mitochondrial biogenesis pathway, and targeting components of this pathway could provide therapeutic opportunities.

Retrospective clinical studies suggest that patients with PDAC whose tumours have a fibrogenic but inert stroma (defined by extensive extracellular matrix (ECM) deposition, low expression of the myofibroblast marker α-SMA and low levels of matrix metalloprotease (MMP) activity) have improved progression-free survival compared to patients whose tumours are populated by a fibrolytic stroma (defined by a low content of collagen fibres, high expression of α-SMA and high levels of MMP activity)⁵. How the stromal state affects clinical out-come is unknown. Moreover, previous investigations of the influence of the stroma on the growth and progression of PDAC have yielded conflicting results, assigning stroma and cancer-associated fibro-blasts (CAFs) as either tumour-supportive⁶ or tumour-restrictive4. It is likely that the failure of stromal-targeted PDAC therapies⁷ is due, in part, to unrecognized pathways that result in tumour-promoting or tumour-suppressive stromal subgroups; successful treatments may thus require precision medicine rather than one-size-fits-all approaches.

cCol I and iCol I Differentially Affect PDAC Growth

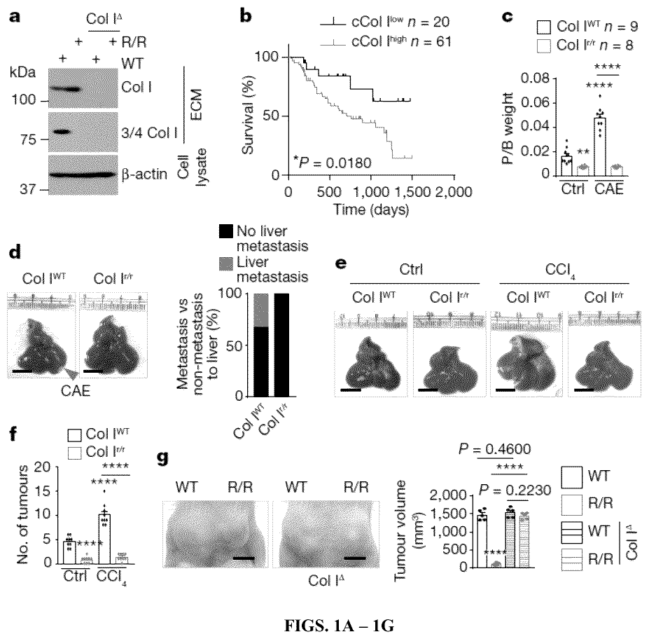

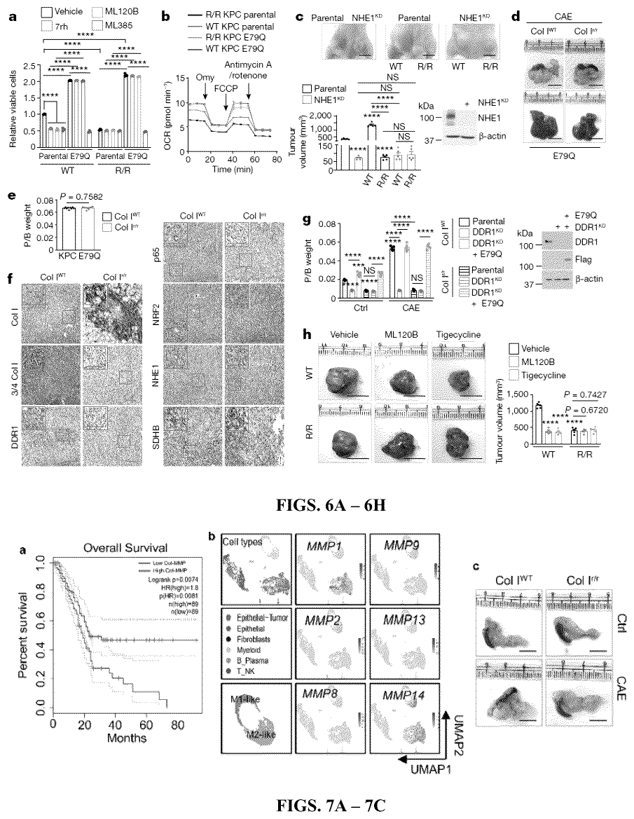

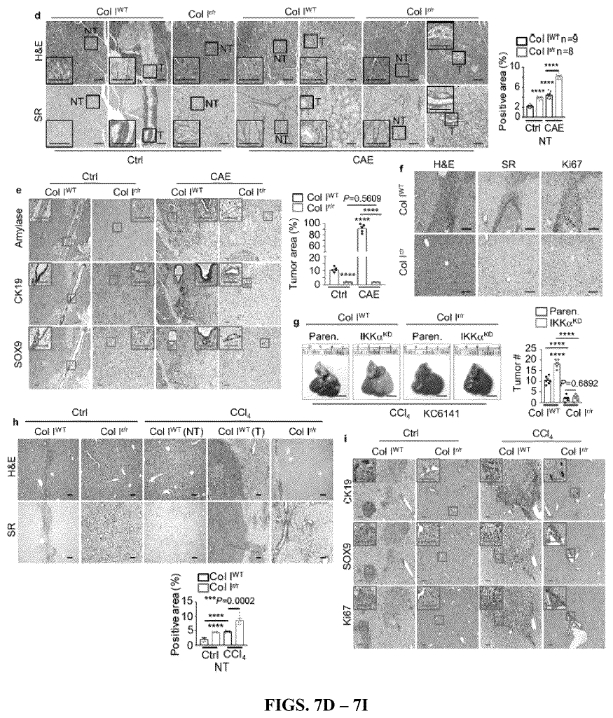

To investigate how the fibrolytic stroma affects PDAC outcome, Applicant compared survival between patients with high and low collagenolysis, using a panel of collagen-cleaving MMPs (MMP1, MMP2, MIMP8, MMP9, MMP13 and MMP14), and found that high mRNA expression of MMPs correlated with poor survival (FIG. 7A). Single-cell RNA sequencing (scRNA-seq) revealed that MMP1, MMP14 and MMP2 mRNAs were the most abundant MMP family members, and were expressed in epithelial-tumour cells, M2-like macrophages and fibroblastic cells (FIG. 7B). The main target of MMPs in desmoplastic tumours is Col I, the prevalent ECM protein. Using antibodies that distinguish iCol I from cCol I (3/4 Col I; FIG. 1A), Applicant stratified a cohort of 106 patients with PDAC whose tumours had been resected, and correlated the tumour Col I state with survival data. These results also pointed to Col I remodeling as a strong prognostic factor, as patients whose tumours were enriched for cCol I had poorer median survival (FIG. 1B). To understand the basis for these results and mimic a cCol I^{low} inert tumour stroma, Applicant used mice expressing either wild-type Colla1^{+/+}(Col I^{WT}), or a MMP-resistant version of Col I generated by two amino acid substitutions in the 1α1 subunit that block the cleavage of Col I by MMPs⁸, Colla1^{r/r} (Col I^{r/r}). Col I^{WT} mice develop more-extensive hepatic fibrosis than Col I^{WT} mice, but despite the hepatocellular carcinoma (HCC)-supportive functions of hepatic fibrosis⁹, they poorly accommodate HCC growth, through unknown mechanisms¹⁰. Col I^{WT} and Col I^{r/r} mice were either orthotopically or intrasplenically (to model liver metastasis) transplanted with mouse PDAC KPC960 (KPC) or KC6141 (KC) cells. Col I^{r/r} mice poorly supported the growth of primary pancreatic tumours or hepatic metastases, even though their pancreata were more fibrotic than Col I^{WT} pancreata. These differences persisted in mice that were pretreated with the pancreatitis inducer caerulein (CAE), which stimulated liver metastasis in Col I^{WT} pancreata (FIG. 1C, FIG. 1D and FIGS. 7C-7F). After intrasplenic transplantation, KPC or KC tumours in Col I^{WT} livers were larger in mice pretreated with CCl₄ to induce liver fibrosis, whereas the number and size of tumours were lower in Col I^{r/r} livers, regardless of CCl₄ pretreatment (FIG. 1E, FIG. 1F and FIG. 7G). As expected, Col I^{r/r} livers were more fibrotic than Col I^{WT} livers, regardless of CCl₄ pretreatment (FIG. 7H). Primary PDAC and liver metastases were confirmed by staining with ductal (CK19), progenitor (SOX9) or proliferation (Ki67) markers (FIG. 7E, FIG. 7F, FIG. 7I). Enhanced tumour growth in CAE- or CCl₄-pretreated Col I^{WT} mice suggested that tumour suppression in Col I^{r/r} mice was not simply due to a space limitation imposed by a build-up of Col I. To determine how Col I remodeling affects human PDAC, Applicant subcutaneously co-transplanted wild-type and R/R fibroblasts with a patient-derived xenograft cell line (1305) into immunocompromised Nu/Nu mice. Wild-type fibroblasts enhanced tumour growth, whereas R/R fibroblasts inhibited tumour growth but lost their inhibitory activity after ablation of Colla1 (FIG. 1G) whose loss did not affect the stimulatory activity of wild-type fibroblasts, suggesting a specific inhibitory function of noncleaved Col I.

The Col I State Controls PDAC Metabolism

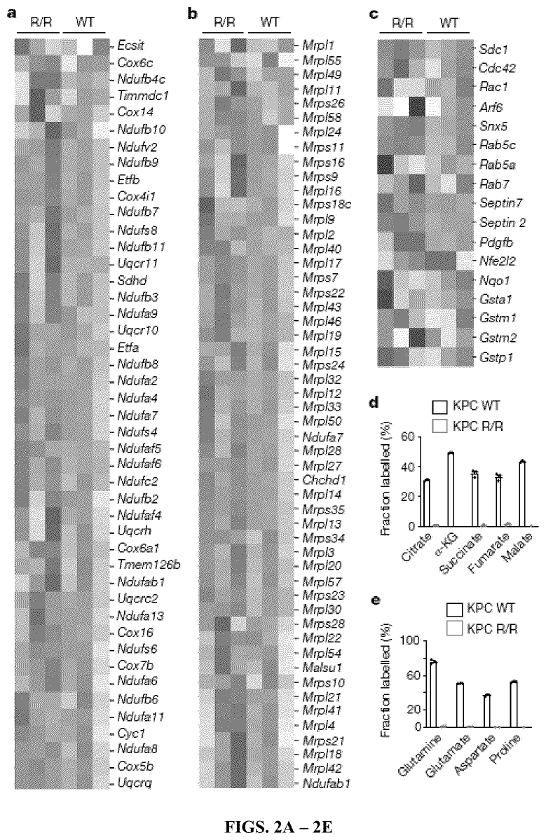

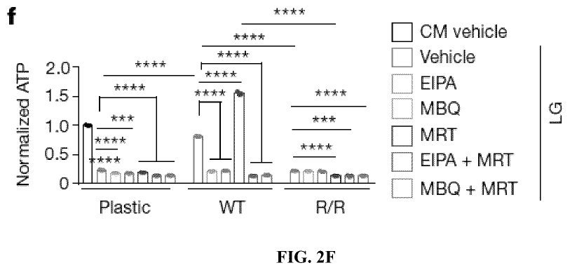

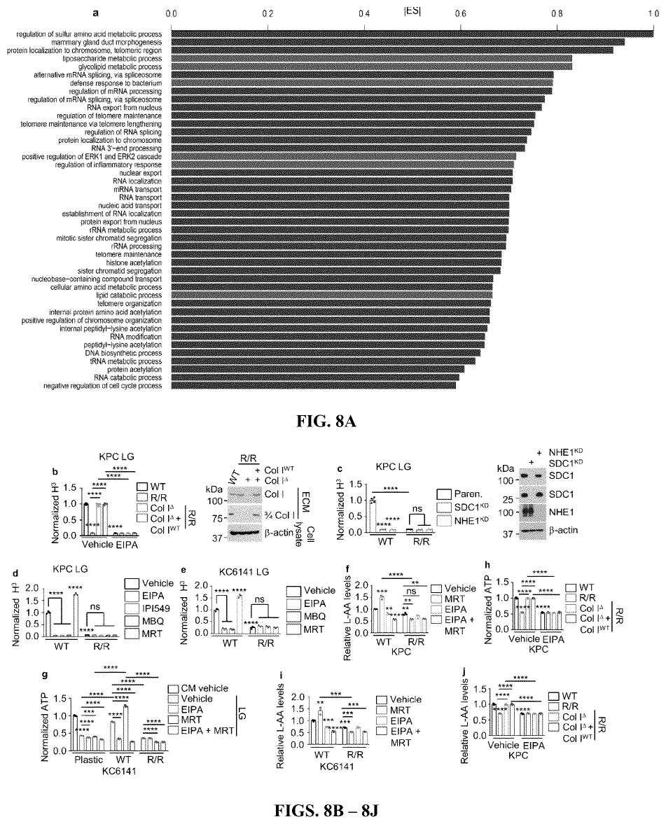

To determine the basis for reduced tumorigenesis in Col I^{r/r} mice, Applicant plated KPC cells on ECM deposited by wild-type and R/R fibroblasts, incubated them in low-glucose (LG) medium (to model nutrient restriction) and performed RNA sequencing (RNA-seq). Bioinformatic analysis revealed marked differences between cells cultured on wild-type and cells cultured on R/R ECM, with the former showing an upregulation of signatures related to sulfur amino acid metabolism, mammary gland morphogenesis, telomere maintenance and RNA processing, and the latter showing an upregulation of mRNAs related to innate immunity and inflammation (FIG. 8A). The most notable differences were in nuclear and mitochondrial genes that encode components of the mitochondrial electron transfer chain (ETC) and ribosome subunits, and macropinocytosis-related genes, which were upregulated by wild-type and suppressed by R/R ECM (FIGS. 2A-2C). Consistent with the upregulation of macropinocytosis-related genes by wild-type ECM, IKKα-deficient KC cells, which have high macropinocytosis activity¹¹, grew better than parental cells in Col I^{WT} livers, but grew as poorly as parental KC cells in Col I^{r/r} livers (FIG. 7G).

To assess the effects of Col I on metabolism, Applicant labeled wild-type and R/R fibroblasts with [³H]-proline or [U-¹³C]-glutamine for five days, during which period the cells coated the plates with Col I-containing ECM. After decellularization, KPC or KC cells and variants thereof were plated and cultured for 24 h in LG medium. The uptake of [³H] in cells plated on wild-type ECM was dependent on macropinocytosis, as indicated by sensitivity to macropinocytosis inhibitors (EIPA (an NHE1 inhibitor), IPI549 (a PI3Kγ inhibitor) or MBQ-167 (a CDC42 and RAC inhibitor)) and to the knockdown of NHE 1 or SDC1, and enhancement by the ULK1 inhibitor MRT68921 (MRT)¹¹. By contrast, cells plated on R/R ECM showed a negligible uptake of [³H] that was unaffected by the inhibition of macropinocytosis (FIGS. 8B-8E). Notably, ablation of Colla1 or overexpression of cleavable Col I in ECM-laying R/R fibroblasts restored [³H] uptake (FIG. 8B). Cells that were cultured on ¹³C-glutamine-labelled wild-type ECM took up glutamine and metabolized it, but cells that were plated on ¹³C-glutamine-labelled R/R ECM exhibited minimal glutamine uptake and metabolism (FIG. 2D, FIG. 2E). Congruently, cells that were cultured on wild-type ECM had higher levels of ATP and a higher amino acid content than cells that were cultured on R/R ECM, and this effect was further increased by treatment with MRT and reduced by blockade of macropinocytosis; by contrast, cells that were cultured on R/R ECM had low levels of ATP and amino acids, which were barely affected by the inhibition of macropinocytosis (FIG. 2F and FIGS. 8F-8J). Ablation of Col I or overexpression of wild-type Col I prevented the decline in ATP and amino acids (FIG. 8H, FIG. 8J), suggesting that cCol I is a key signaling molecule that stimulates PDAC metabolism and energy generation.

cCol I to iCol I Ratio Controls DDR1—NRF2 Signaling

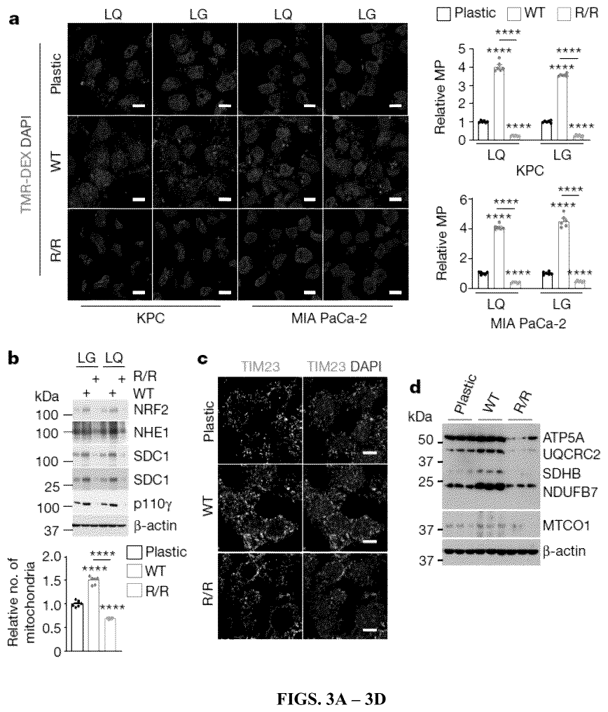

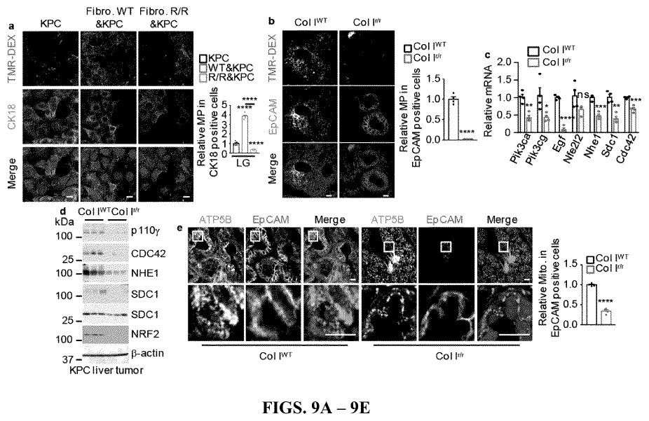

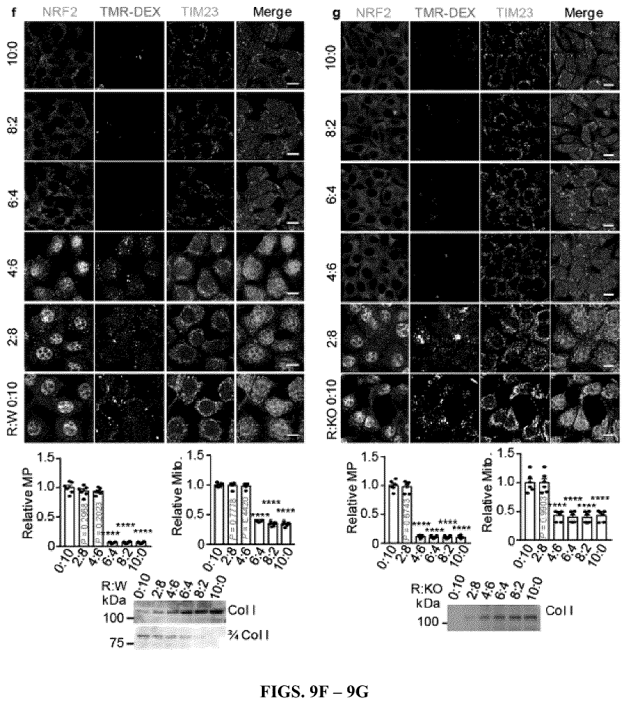

KPC or human MIA PaCa-2 cells plated on wild-type ECM or co-cultured with wild-type fibroblasts in LG or low-glutamine (LQ) medium exhibited high rates of macropinocytosis, as measured by their uptake of tetramethylrhodamine-labelled high-molecular-mass dextran (TMR-DEX), whereas cells plated on R/R ECM or co-cultured with R/R fibroblasts exhibited low rates of macropinocytosis (FIG. 3A and FIG. 9A). Furthermore, KPC cells cultured on wild-type ECM had the opposite effect (FIG. 3B). Similar differences in micropinocytosis activity, NRF2 and micropinocytosis-related mRNAs and proteins were shown by KPC tumours in Col I^{WT} or Col I^{r/r} pancreata or livers (FIGS. 9B-9D). Mitochondria are important for cancer growth in that they generate energy for macromolecular synthesis¹². Consistent with the RNA-seq data, mitochondria and ETC proteins were decreased in PDAC cells grown on R/RECM or in Col I^{r/r} pancreata (FIG. 3C, FIG. 3D and FIG. 9E).

The human PDAC stroma consists of intact and cleaved collagens. To recapitulate this setting and determine how the balance of iCol I to cCol I affects PDAC metabolism, Applicant mixed R/R fibroblasts with wild-type (R:W) or Col I^{Δ} (knockout) (R:KO) fibroblasts to generate ECM with different amounts of iCol I and cCol I, and confirmed this with isoform-specific antibodies. KPC cells were plated on the ECM preparations and kept in LG medium for 24 h, and their rates of macro-pinocytosis, numbers of mitochondria and levels of nuclear NRF2 were evaluated. Nondegradable Col I at 6:4 (R:W) or 4:6 (R:KO) ratios and higher ratios inhibited macropinocytosis and reduced mitochondria numbers and nuclear NRF2 (FIG. 9F, FIG. 9G). Applicant conclude that iCol I inhibits macropinocytosis and mitochondrial biogenesis, which are stimulated by different cleaved collagens, not just cCol I.

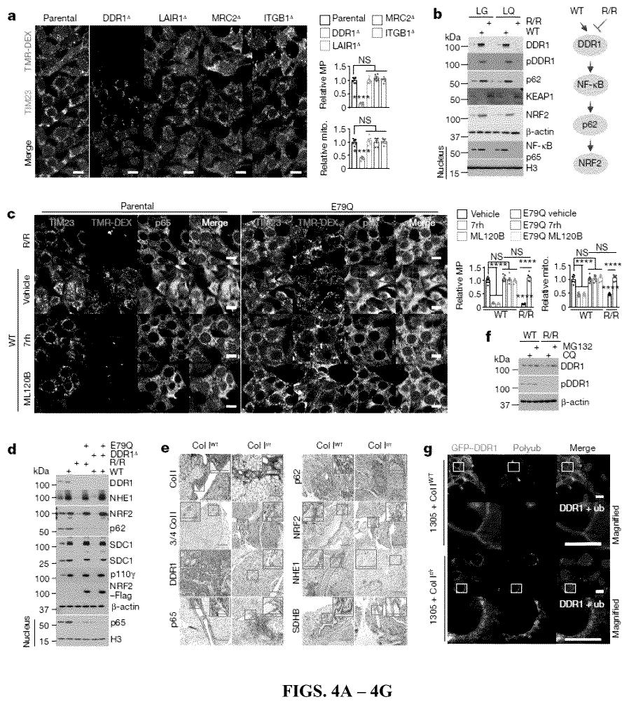

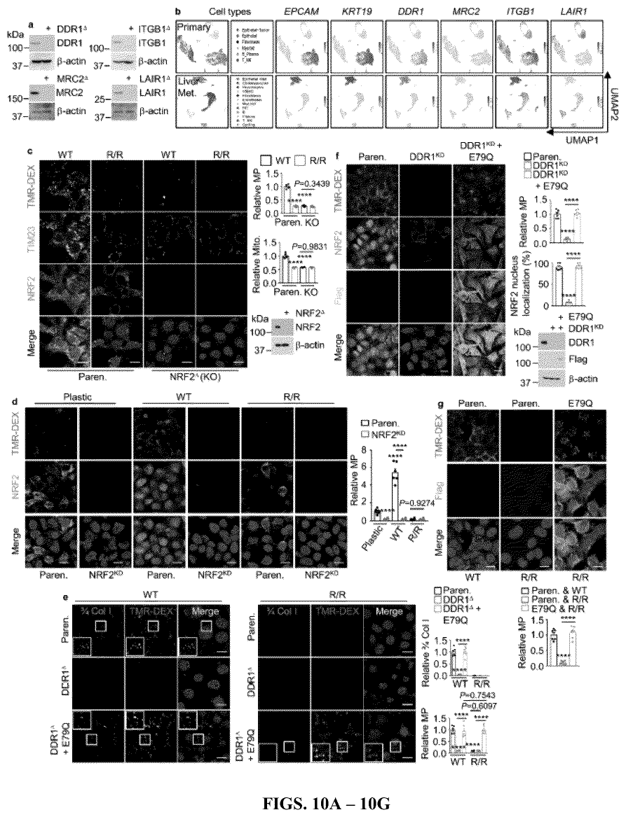

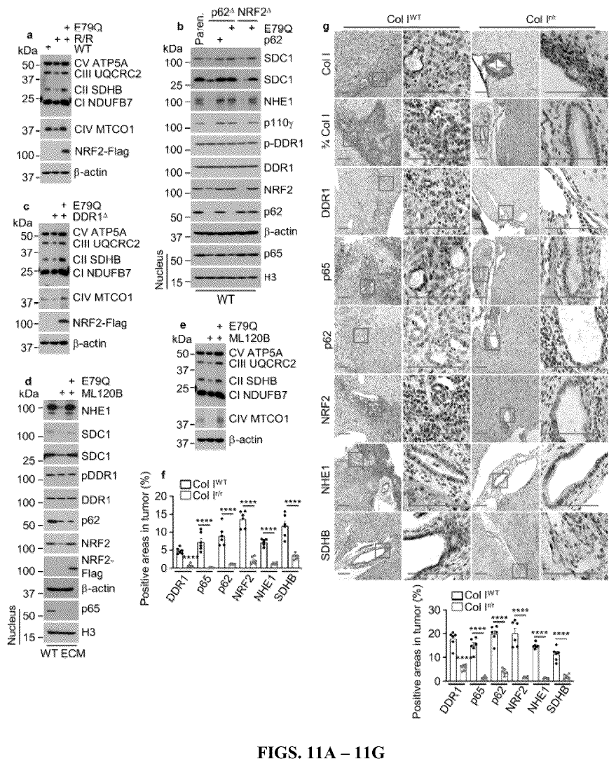

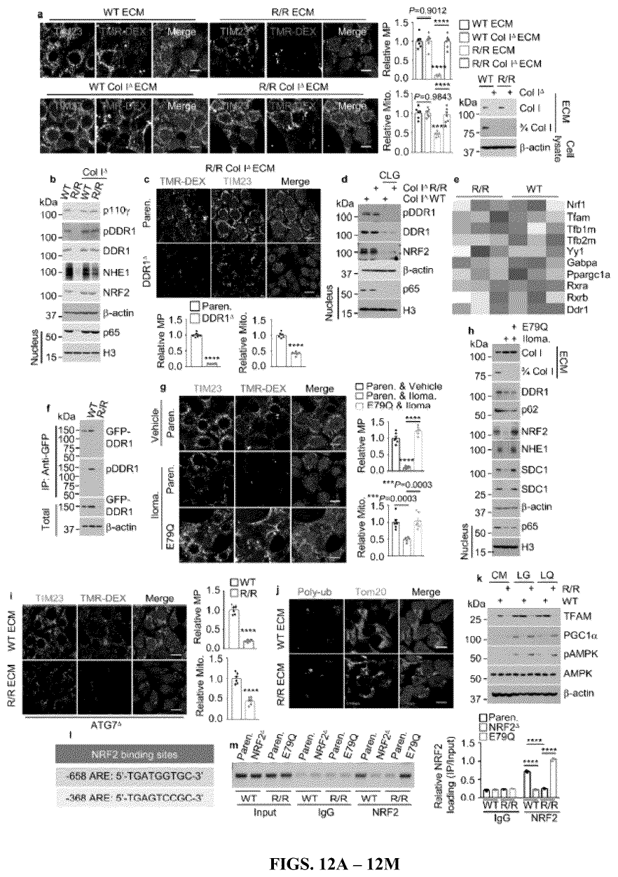

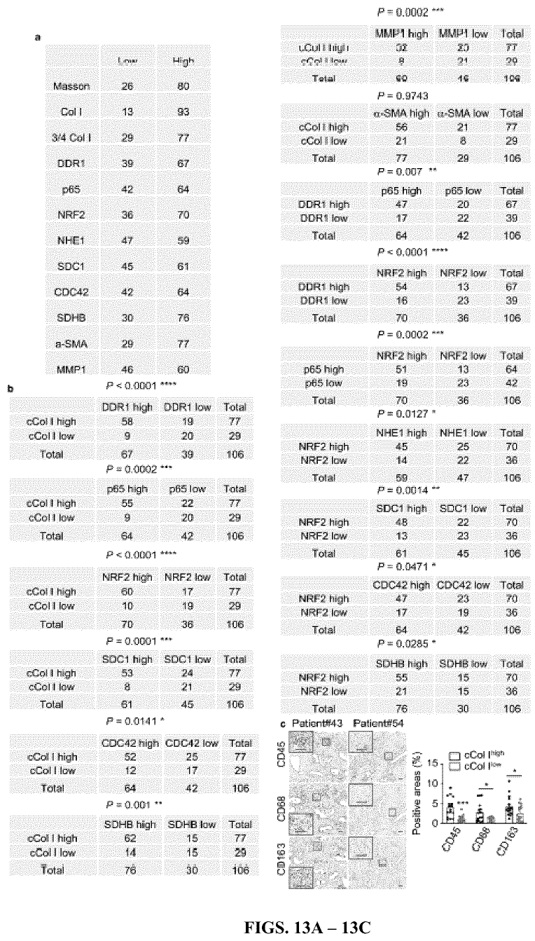

To investigate how Col I regulates macropinocytosis and mitochondrial biogenesis, Applicant systematically ablated (FIG. 10A) all known collagen receptors expressed by KPC cells—MRC2, DDR1, LAIR1 and β1 integrin (ITGB1). The only receptor whose ablation inhibited macropinocytosis activity and mitochondrial biogenesis (FIG. 4A) was DDR1, a collagen-activated receptor tyrosine kinase (RTK)¹³, which scRNA-seq showed was highly expressed in primary and liver-metastatic human PDAC epithelial-tumour cells, marked by the mRNA expression of EPCAM and KRT19 (FIG. 10B). Other collagen receptor mRNAs were either not expressed in PDAC (LAIR1 and MRC2) or had a broad distribution (ITGB1). Whereas wild-type ECM stimulated the expression and phosphorylation of DDR1, R/R ECM strongly downregulated DDR1 and its downstream effector NF-κ¹⁴, as well as p62 (FIG. 4B), an NF-βB target's. The inhibitory effect of iCol I was not observed in previous DDR1 signaling studies, which used artificially fragmented acid-solubilized collagens as ligands¹⁶. Consistent with the induction of p62, wild-type ECM decreased KEAP1 and upregulated NRF2, whereas R/R collagen had the opposite effect (FIG. 4B). Applicant questioned whether cCol I affects macropinocytosis and mitochondrial biogenesis through the DDR1—NF-κB—p62—NRF2 cascade. Indeed, R/R ECM and inhibition or ablation of NRF2, DDR1 or IKKβ decreased macropinocytosis activity, 3/4 Col I fragment uptake, NRF2 nuclear localization, mitochondria number and expression of macropinocytosis-related and mitochondrial ETC proteins (FIG. 4C, FIG. 4D and FIGS. 10C-10G and FIGS. 5A-5E). Overexpression of an activated NRF2(E79Q) variant reversed the inhibitory effects of R/R ECM, DDR1 inhibition or IKKO inhibition but did not restore or affect DDR1 expression or phosphorylation and p65 nuclear localization. Consistent with these data, pancreatic and liver tumours from Col I^{r/r} mice showed more-extensive expression of iCol I but no cCol I and lower levels of DDR1, p65, p62, NRF2, NHE1 and SDHB (a mitochondrial marker), as compared to tumours from Col I^{WT} mice (FIG. 4E and FIG. 11F, FIG. 11G). These results suggest that Col I controls macropinocytosis and mitochondrial biogenesis through the DDR1—NF-κB—p62—NRF2 axis. As myofibroblast-specific ablation of Col I enhances intrahepatic PDAC growth17, Applicant examined how Col I^{Δ} ECM affects macropinocytosis and DDR1 signaling. Notably, Col I^{Δ} ECM behaved like wild-type ECM, stimulating macropinocytosis, mitochondrial biogenesis and DDR1 phosphorylation, which were blocked by the ablation of DDR1 (FIGS. 12A-12C). However, collagen-free ECM generated by Col I^{Δ} fibroblasts and treatment with bacterial collagenase no longer activated DDR1 and its downstream effectors (FIG. 12D). These results are consistent with DDR1 being a general collagen receptor¹³, with other collagens in Col I^{Δ} fibroblasts acting as ligands.

iCol I Triggers DDR1 Proteasomal Degradation

The expression and function of DDR1 vary in different cancer stages and types¹⁸⁻²¹. Levels of mouse Ddr1 mRNA were increased by culturing KPC cells on R/R ECM (FIG. 12E), implying that the diminished expression of DDR1 protein in these cultures is post-transcriptional. Indeed, MG132, a proteasome inhibitor, but not the lysosomal inhibitor chloroquine, rescued DDR1 expression but not autophosphorylation (FIG. 4F). Notably, GFP—DDR1 showed cell-surface localization and little polyubiquitin colocalization in human 1305 cells that were co-cultured with wild-type fibroblasts, but was cytoplasmic and colocalized with polyubiquitin in R/R fibroblast cocultures (FIG. 4G). Unlike DDR1 in triple-negative breast cancer (TNBC)²⁰, no shedding of the DDR1 extracellular domain was detected (FIG. 12F). Applicant's results therefore reveal a new mode of DDR1 regulation in PDAC and probably in other desmoplastic cancers.

NRF2 Controls Mitochondrial Biogenesis

ECM from fibroblasts treated with the FDA-approved MMP inhibitor Ilomastat behaved like R/R ECM (FIG. 12G, FIG. 12H), indicating that the results were not unique to the Col I^{R} variant. R/R ECM also decreased the number of mitochondria in autophagy-deficient PDAC cells (FIG. 12I), which suggests that the reduced mitochondrial content is not mediated by mitophagy. Moreover, colocalization of mitochondria and polyubiquitin, which marks mitophagy, was rarely observed (FIG. 12J). Expression of TFAM, a key activator of mitochondrial DNA transcription, replication and biogenesis²², was downregulated in PDAC cells cultured in R/R ECM, but Nrf1 (unrelated to NRF2) mRNA, PGC1α protein and AMPK activity, which also stimulate mitochondrial biogenesis²³, were upregulated (FIG. 12E, FIG. 12K). The latter results match the low ATP content of R/R-ECM-cultured cells. In silico analysis revealed putative NRF2-binding sites in the Tfam promoter region, to which NRF2 was recruited in cells plated on wild-type ECM or in NRF2(E79Q)-expressing cells (FIG. 12L, FIG. 12M), confirming that NRF2 mediates cCol I-stimulated macropinocytosis and mitochondrial biogenesis.

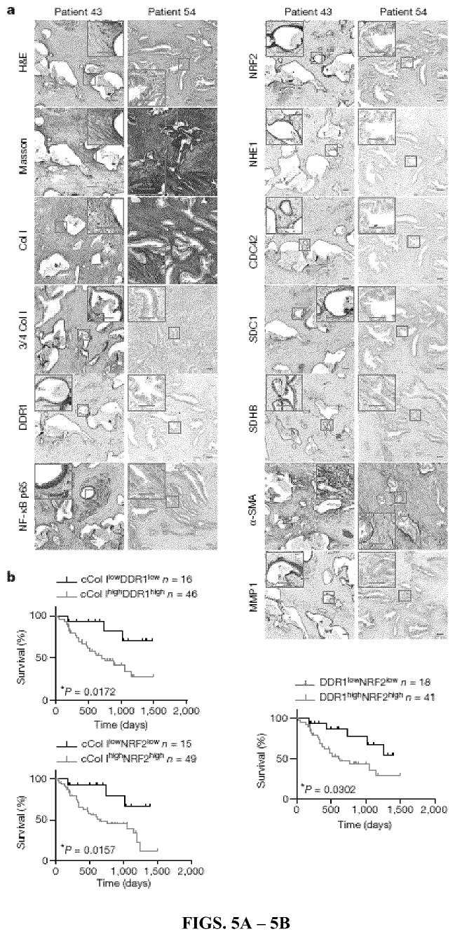

Higher levels of iCol I correlate with improved survival Immunohistochemistry (IHC) of surgically resected human PDAC showed that most tumours (77/106) contained high amounts of 3/4 Col I and most of them exhibited higher levels of staining for DDR1 (58/77), NF-κB p65 (55/77), NRF2 (60/77), SDC1 (53/77), CDC42 (52/77), SDHB (62/77), α-SMA (56/77) and MIMP1 (52/77) than did cCol I^{low} tumours (FIG. 5A and FIG. 13A, FIG. 13B), suggesting that PDAC tumours with fibrolytic stroma have higher macropinocytosis activity and mitochondrial content than do tumours with inert stroma. Moreover, DDR1 and p65, DDR1 and NRF2, p65 and NRF2, NRF2 and macropinocytosis proteins (NHE1, SDC1 or CDC42), and NRF2 and SDHB showed strong positive correlations (FIG. 13B), suggesting that the fibrolytic stroma stimulates macropinocytosis and mitochondrial biogenesis through the DDR1—NF-κB—NRF2 axis in human PDAC. Increased levels of cCol I also correlated with high expression of inflammatory markers (FIG. 13C), supporting the notion that inflammation may drive Col I remodeling. Notably, patients with cCol I^{high} and DDR1^{high}, cCol I^{high} and NRF2^{high} or DDR1^{high} and NRF2^{high} tumours had a considerably worse median survival than did patients with low expression of these markers (FIG. 5B). These results are consistent with those obtained in Applicant's preclinical PDAC models, suggesting that the fibrolytic stroma may drive the recurrence of human PDAC through NRF2-mediated macropinocytosis and mitochondrial biogenesis.

Targeting the DDR1—NF-κB—NRF2 Cascade

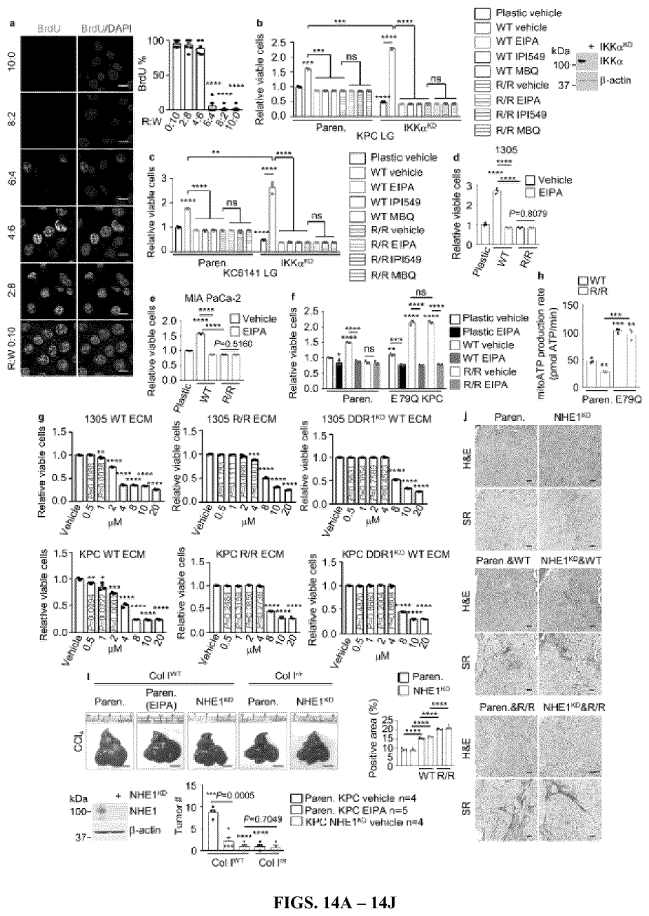

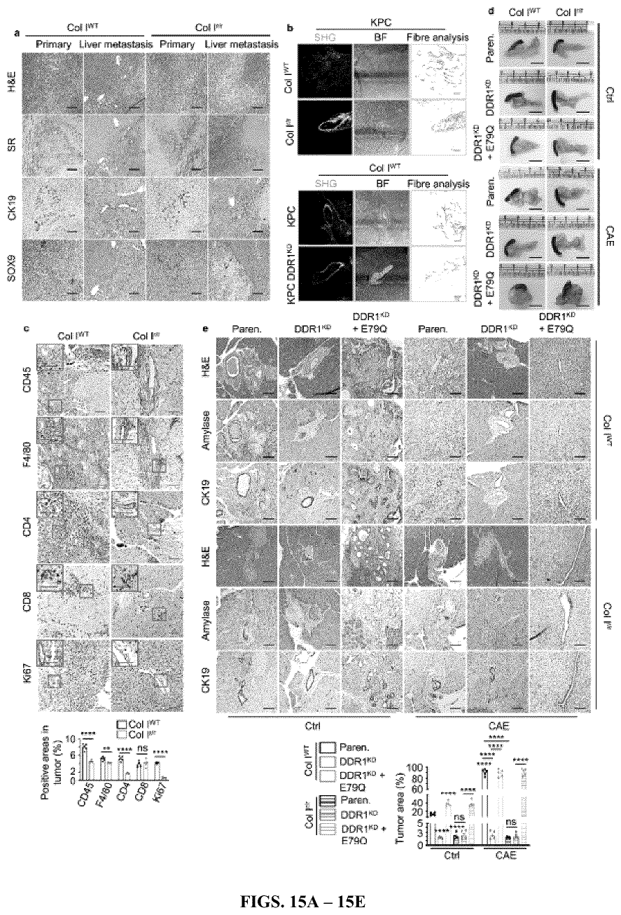

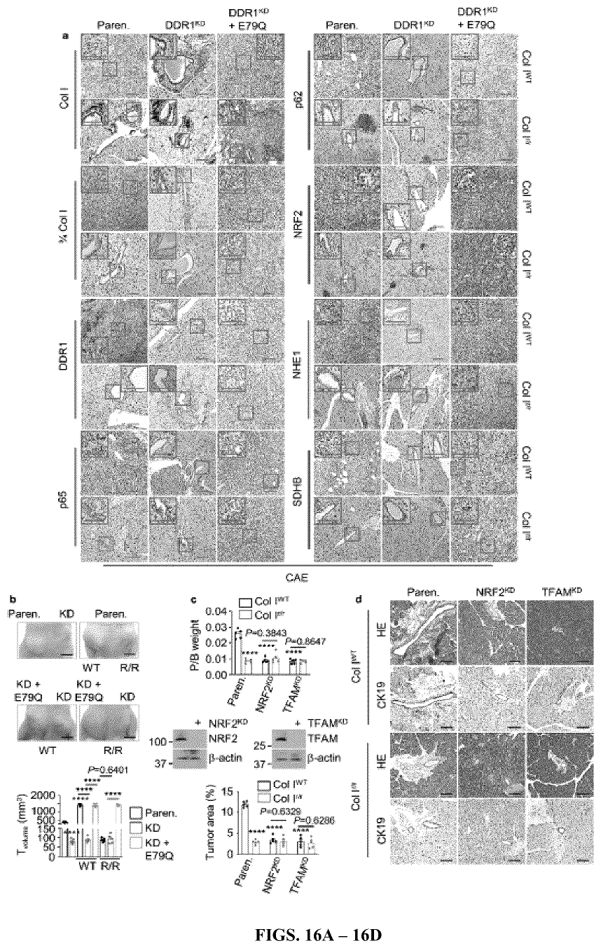

Increasing iCol I in the ECM inhibited cellular DNA synthesis (FIG. 14A). Parental, NRF2^{E79Q} or IKKα-knockdown (IKKα^{KD}) PDAC cells were plated on wild-type or R/R ECM, incubated in LG medium and treated with inhibitors of DDR1 (7rh), IKKfβ (ML120B), NRF2 (ML385) or macropinocytosis (NRE1^{KD} or EIPA, IPI549 or MBQ-167). Whereas wild-type ECM increased and R/R ECM decreased parental PDAC cell growth, inhibition of macropinocytosis, DDR1, IKKβ or NRF2 decreased growth on wild-type ECM (FIG. 6A and FIGS. 14B-14F). NRF2(E79Q)-expressing cells grew faster than parental cells and were resistant to R/R ECM, DDR1 inhibition or IKKβ inhibition but not NRF2 inhibition. IKKα^{KD} cells with high rates of macropinocytosis and high levels of nuclear NRF2 also grew faster than parental cells on wild-type ECM but were more sensitive to R/R ECM and macropinocytosis inhibitors (FIG. 14B, FIG. 14C). Inhibition of macropinocytosis, DDR1, IKKβ or NRF2 did not decrease the low growth of parental cells on R/R ECM (FIG. 6A and FIGS. 14B-14F). Moreover, parental KPC or 1305 cells that were plated on wild-type ECM were more sensitive to the mitochondrial protein synthesis inhibitor tigecycline than cells plated on R/R ECM or DDR1^{KD} cells grown on wild-type ECM (FIG. 14G). NRF2^{E79Q} cells showed higher rates of oxygen consumption and mitochondrial ATP production than did parental cells; these rates were diminished by R/R ECM but only in the parental cells (FIG. 6B and FIG. 14H). Thus, the fibrolytic stroma may support PDAC cell growth through Col I-stimulated macropinocytosis and mitochondrial biogenesis. R/R fibroblasts inhibited human PDAC (MIA PaCa-2) tumour growth, but wild-type fibroblasts were stimulatory. NHE1 ablation or EIPA inhibited tumour growth with or without co-transplanted wild-type fibroblasts or in wild-type livers, but had little effect on tumours growing with R/R fibroblasts or in Col I^{r/r} livers (FIG. 6C and FIG. 14I). Tumours growing with wild-type fibroblasts were more fibrotic than tumours without added fibroblasts, and small tumours growing with R/R fibroblasts had the highest collagen content (FIG. 14J), indicating that deposition of Col I enhances the growth of PDAC only when Col I is cleaved by MMPs. NRF2^{E79Q} cells in Col I^{r/r} hosts exhibited similar growth, NRF2, NHE1 and SDHB expression and liver metastases to cells growing in Col I^{WT} hosts, despite low expression of DDR1 and p65 (FIGS. 6D-6F and FIG. 15A).

In TNBC, DDR1 aligns collagen fibres to exclude immune cells²⁰. By measuring second-harmonic generation (SHG), Applicant observed no change in collagen fibre alignment and CD8⁺ T cell content between tumours from Col I^{WT} and Col I^{r/r} pancreata or between parental and DDR1^{KD} tumours, although CD45-, F4/80- or CD4-expressing cells were reduced in tumours from Col I^{r/r} pancreata (FIG. 15B, FIG. 15C). Accordingly, ablation of DDR1 inhibited tumour growth, p65, p62, NRF2, NHE1 and SDHB expression in Col I^{WT} pancreata but did not reduce it further in Col I^{r/r} pancreata (FIG. 6G and FIG. 15D, FIG. 15E and FIG. 16A). NRF2(E79Q) rescued tumour growth and the expression of NHE1 and SDHB—but not p65 or p62—in DDR1^{KD} cells, regardless of Col I status. Similar results were observed in immunodeficient mice (FIG. 16B), indicating that the effects of Col I-DDR1 interaction differ between PDAC and TNBC. Notably, inhibition of IKKβ, mitochondrial proteinsynthesis, TFAM or NRF2 decreased the growth of tumours that were co-transplanted with wild-type fibroblasts or grown in Col I^{WT} pancreata, but had no effect on tumours that were co-transplanted with R/R fibro-blasts or grown in Col I^{r/r} pancreata (FIG. 6H and FIG. 16C, FIG. 16D), illustrating different ways of targeting PDAC with fibrolytic stroma.

Discussion

Applicant shows here that Col I remodeling is a prognostic indicator for the survival of patients with PDAC. In preclinical models, Col I remodeling modulated tumour growth and metabolism through a DDR1-NF-κB- p62-NRF2 cascade that is activated by cCol I and inhibited by iCol I. The activation of DDR1 by collagens and downstream activation of NF-κB have been described before^{14,16}. However, it was previously unknown—to Applicant's knowledge—that iCol I triggers the polyubiquitylation and proteasomal degradation of DDR1. This indicates that DDR1 distinguishes cleaved from intact collagens, and that the latter are capable of restraining the metabolism and growth of tumours. Although inhibition of DDR1 reduces the growth of mouse PDAC²⁴, the ability of DDR1 to control tumour metabolism by stimulating macropinocytosis and mitochondrial biogenesis was unknown. It is unclear, however, why DDR1—a rather weak RTK¹³—exerts such profound metabolic effects on PDAC cells that express more potent RTKs, such as EGFR and MET. Perhaps this is due to high concentrations of cCol I in the PDAC tumour microenvironment and the stronger NF-κB-activating capacity of DDR1 relative to other RTKs. Indeed, IKKβ inhibition was as effective as the blockade of mitochondrial protein synthesis in curtailing the growth of PDAC with fibrolytic stroma. The differential effects of fibrolytic and inert tumour stroma on PDAC growth and metabolism explain much of the controversy that surrounds the effects of CAFs and Col I on the progression of PDAC in mice^{6,17}. Most notably, Applicant's findings extend to humans and suggest that Col I remodeling is linked to tumour inflammation. Applicant thus proposed that treatments that target DDR1—IKKβ-NF-κB-NRF2 signaling and mitochondrial biogenesis should be evaluated in prospective clinical trials that include stromal state—an important modifier of tumour growth—as an integral biomarker. Given that three Col I-cleaving MMPs were highly expressed in the human PDAC samples Applicant analysed, and that this situation may differ from patient to patients²⁵, specific MMP inhibitors are additional candidates for precision therapy. Although these results do not apply to TNBC, they provide mechanistic insight into SPARC-mediated PDAC progression^{26, 27}, and can be applicable to other desmoplastic and fibrolytic cancers.

### Materials and Methods

Cell Culture

All cells were incubated at 37° C. in a humidified chamber with 5% CO₂. MIA PaCa-2 (MIA), UN-KPC-960 (KPC) and UN-KC-6141 (KC) cells, wild-type and R/R fibroblasts were maintained in Dulbecco's modified Eagle's medium (DMEM) (Invitrogen) supplemented with 10% fetal bovine serum (FBS) (Gibco). MIA cells were purchased from ATCC. KPC and KC cells were generated at the laboratory of S. K. Batra²⁸. Wild-type and R/R fibroblasts were generated at the laboratory of D.B.¹⁰. The 1305 primary human PDAC cells were generated by the A.M.L. laboratory from a human PDAC patient-derived xenograft¹¹ and were maintained in RPMI (Gibco) supplemented with 20% FBS and 1 mM sodium pyruvate (Corning). All media were supplemented with penicillin (100 mg ml⁻¹) and streptomycin (100 mg ml⁻¹). All cells were partially authenticated by visual morphology. Wild-type and R/R fibroblasts were partially authenticated by ECM production and collagen type I alpha 1 cleavage. KPC and KC cells were partially authenticated by orthotopic tumour formation in mouse pancreas. MIA and 1305 cells were partially authenticated by subcutaneous tumour formation in nude mice. Cells were not further authenticated. Cell lines were tested for mycoplasma contamination. LG medium: glucose-free DMEM medium was supplemented with 0.5 mM glucose in the presence of 10% dialysed FBS and 25 mM HEPES. LQ medium: glutamine-free DMEM medium was supplemented with 0.2 mM glutamine in the presence of 10% dialysed FBS and 25 mM HEPES.

Plasmids

For gene ablations, the target cDNA sequences (Table 1) of mouse Ddr1, Mrc2, Itgb1, Lair1, Nrf2, Colla1 and human DDR1 were cloned into a lentiCRISPR v2-Blast vector or lentiCRISPR v2-puro vector, respectively using BsmBI. For gene knockdowns, pLKO.1-puro-Ddr1 (TRCN0000023369), pLKO.1-puro-DDR1 (TRCN0000121163), pLKO.1-puro-Sdc1 (TRCN0000302270), pLKO.1-puro-Nrf2 (TRCN0000054658) and pLKO.1-puro-Tfam (TRCN0000086064) were ordered from Sigma. pCDH-CMV-MCS-EF1-puro-Collα1-6XHis and pLVX-IRES-Puro-NRF2^{E79Q}-Flag were made by Sangon Biotech (Shanghai, China). pLKO.1-blast-Ikkα, pLKO.1-puro-Nhe1, pLKO.1-puro-NRE1, pLKO.1-puro-NRF2, and lentiCRISPR v2-Puro-p62/Sqstm1 have been described previously¹¹. LentiCRISPR v2-Blast-ATG7 (ref.²⁹) was a gift from S. Ghaemmaghami.

TABLE 1
CRISPR CAS9 sgRNA Sequence List

| Gene | sgRNA sequence (5′-3′) |
|---|---|
| Ddr1 | GTAACGCAACCGATAGCTTC |
| Mrc2 | CCGGTGGACCAATGTCAAGG |
| Itgb1 | AATGTCACCAATCGCAGCAA |
| Lair1 | GTCCGAACGTAGTAAGACGC |
| Nrf2 | GGCATCTTGTTTGGGAATGT |
| Col1α1 | CGTGCAATGCAATGAAGAAC |
| DDR1 | GGATCTACAACGACTGCACC |

Stable Cell Line Construction

Lentiviral particles were generated as before³⁰. MIA, 1305, KPC or KC cells and fibroblasts were transduced by combining 1 ml of viral particle-containing medium with 8 μg ml⁻¹ polybrene. The cells were fed 8 h later with fresh medium and selection was initiated 48 h after trans-duction using 1.25 μg ml⁻¹ puromycin or 10 μg ml⁻¹ blasticidin. IKKα^{KD} KC, NRF2^{KD} MIA and ATG7^{Δ} MIA cells have been described previously¹¹.

Mice

Female homozygous^{Nu/Nu} nude mice and C57BL/6 mice were obtained at six weeks of age from Charles River Laboratories and The Jackson Laboratory, respectively. Colla1^{+/+} (Col I^{WT}) or Colla1^{r/r} (Col I^{r/r}) mice on a C57BL/6 background were obtained from D.B. at UCSD and were previously described^{8, 31}. Mice matched for age, gender and equal average tumour volumes were randomly allocated to different experimental groups on the basis of their genotypes. No sample size pre-estimation was performed but as many mice per group as possible were used to minimize type I/II errors. Both male and female mice were used unless otherwise stated. Blinding of mice was not performed except for IHC analysis. All mice were maintained in filter-topped cages on autoclaved food and water at constant temperature and humidity and in a pathogen-free controlled environment (23° C.±2° C., 50-60%) with a standard 12-h light-12-h dark cycle. Experiments were performed in accordance with UCSD Institutional Animal Care and Use Committee and NIH guidelines and regulations. Animal protocol S00218 (M.K.) was approved by the UCSD Institutional Animal Care and Use Committee. The number of mice per experiment is indicated in the figure legends and their age is indicated in Methods.

Orthotopic PDAC Cell Implantation

Col I^{WT} or Col I^{r/r} mice were pretreated with or without 50 μg kg⁻¹ CAE by intraperitoneal injections every hour, six times daily on the first, fourth and seventh days. On day 11, parental, NRF2^{E79Q}, DDR1^{KD}, DDR1^{KD}+NRF2^{E79Q}, NRF2^{KD} or TFAMKD KPC or KC cells were orthotopically injected into three-month-old Col I^{WT} or Col I^{r/r} mice as described¹¹. After surgery, mice were given buprenorphine subcutaneously at a dose of 0.05-0.1 mg kg⁻¹ every 4-6 h for 12 h and then every 6-8 h for 3 additional days. Mice were analysed after four weeks.

Intrasplenic PDAC Cell Implantation

Three-month-old Col I^{WT} or Col I^{r/r} mice were treated with or without an oral gavage of 25% CCl₄ in corn oil twice a week for two weeks. After two weeks of recovery, parental, NHE1^{KD} or IKKα^{KD} KPC or KC cells (10⁶ cells in 50 μl phosphate-buffered saline; PBS) were adoptively transferred into the livers of Col I^{WT} or Col I^{r/r} mice by intrasplenic injection, followed by immediate splenectomym. Mice were analysed 14 days after treatment with or without 10 mg kg⁻¹ EIPA (Sigma) by intraperitoneal injection every other day.

Subcutaneous PDAC Cell Implantation

Homozygous BALB/c Nu/Nu female mice were injected subcutaneously in a single flank or in both flanks at 7 weeks of age with 5×10⁵ parental, NHE1^{KD}, DDR1^{KD} or DDR1^{KD}+NRF2^{E79Q} MIA cells or 1305 cells mixed with or without 5×10⁵ wild-type, R/R, Col I^{Δ} wild-type or Col I^{Δ} R/R fibroblasts diluted 1:1 with BD Matrigel (BD Biosciences) in a total volume of 100 μl. Tumours were collected after four weeks. To evaluate the effect of IKKβ or mitochondrial protein synthesis inhibition on tumour growth, mice were treated with vehicle (dimethyl sulfoxide in PBS), ML120B (60 mg kg⁻¹) twice daily through oral gavage or tigecycline (50 mg kg⁻¹) twice daily through intraperitoneal injection for three weeks. Therapy was started one week after tumour implantation. Volumes (½×(width×length)) of subcutaneous tumours were calculated on the basis of digital caliper measurements. Mice were euthanized to avoid discomfort if the tumour diameter reached 2 cm.

Samples of Human PDAC

Survival analysis of patients expressing high and low levels of Col I—MMP was performed using The Cancer Genome Atlas (TCGA) data and the GEPIA2 platform. The collagen-cleaving signature consisted of MMP1, MMP2, MIMP8, MMP9, M MP13 and MMP14. Overall survival was determined in the TCGA cohort of 178 patients with PDAC using a median cut-off.

A total of 106 specimens of human PDAC were acquired from patients who were diagnosed with PDAC between January 2017 and May 2021 at The Affiliated Drum Tower Hospital of Nanjing University Medical School. All patients received standard surgical resection and did not receive chemotherapy before surgery. Paraffin-embedded tissues were processed by a pathologist after surgical resection and confirmed as PDAC before further investigation. Overall survival duration was defined as the time from the date of diagnosis to that of death or last known follow-up examination. Survival information was available for 81 of the 106 patients. The study was approved by the Institutional Ethics Committee of The Affiliated Drum Tower Hospital with IRB 2021-608-01. Informed consent for tissue analysis was obtained before surgery. All research was performed in compliance with government policies and the Helsinki declaration.)

IHC

Pancreata or liver were dissected and fixed in 4% paraformaldehyde in PBS and embedded in paraffin. Five-micrometre sections were prepared and stained with H&E or sirius red. IHC was performed as before¹¹. Slides were photographed on an upright light/fluorescent Imager A2 micro-scope with AxioVision Rel. 4.5 software (Zeiss). Antibody information is shown in Table 2.

IHC Scoring

IHC scoring was performed as before¹¹. Negative and weak staining was viewed as a low expression level and intermediate and strong staining was viewed as a high expression level. For cases with tumours with two satisfactory cores, the results were averaged; for cases with tumours with one poor-quality core, results were based on the interpretable core. On the basis of this evaluation system, a chi-squared test was used to estimate the association between the staining intensities of Col I—DDR1—NRF2 signaling proteins. The number of evaluated cases for each different staining in PDAC tissues and the scoring summary are indicated in FIG. 13A.

ECM preparation

Wild-type or R/R fibroblasts were seeded on 6, 12 or 96-well plates. One day after plating, cells were switched into DMEM (with pyruvate) with 10% dialysed FBS supplemented with or without 500 μM [³H]-proline or [U-¹¹C]-glutamine and 100 μM vitamin C. Cells were cultured for five days with renewal of the medium every 24 h. Then fibroblasts were removed by washing in 1 ml or 500 μl or 100 μl per well PBS with 0.5% (v/v) Triton X-100 and 20 mM NH4OH. The ECM was washed five times with PBS before cancer cell plating. The following day, cancer cells were switched into the indicated medium for 24 or 72 h.

Cell imaging

Cells were cultured on coverslips coated with or without ECM and fixed in 4% paraformaldehyde for 10 min at room temperature or methanol for 10 min at —20° C. Macropinosome visualization in cell and tissue and immunostaining were performed as previously described¹¹. Images were captured and analysed using a TCS SPE Leica confocal microscope with Leica Application Suite AF 2.6.0.7266 software (Leica). Antibody information is shown in Table 2.

SHG

Mouse pancreatic tumour tissue was fixed in 4% paraformaldehyde in PBS and embedded in paraffin. Five-micrometre sections were prepared and deparaffinized in xylene, rehydrated in graded ethanol series as described³², mounted using an aqueous mounting medium and sealed with a coverslip. All samples were imaged using a Leica TCS SP5 multiphoton confocal microscope and an HC APO LC 20×1.00W was used throughout the experiment. The excitation wavelength was tuned to 840 nm, and a 420±5-nm narrow bandpass emission controlled by a prism was used for detecting the SHG signal of collagen. SHG signal is generated when two photons of incident light interact with the non-centrosymmetric structure of collagen fibres, which leads to the resulting photons being half the wavelength of the incident photons. SHG measurements were performed using CT-Fire software (v.2.0 beta) (https://loci.wisc.edu/software/ctfire). The tumour area was confirmed by H&E staining.

Immunoblotting and immunoprecipitation

Preparation of protein samples from cells and tissues, immunoblotting and immunoprecipitation were performed as before^{10, 30}. Immunoreactive bands were detected by an automatic X-ray film processor or a KwikQuant Imager. Antibody information is shown in Table 2.

TABLE 2
Antibody List

| Antibody | Catalogue Number | Company |
|---|---|---|
| anti-p62 | GP62-C | Progen |
| anti-NRF2 | A11159 | ABclonal |
| anti-COL1A1 | 72026 | CST |
| | sc-293182 | Santa Cruz |
| anti-3/4 COL1A1 | 0217-050 | Immunoglobe |
| anti-TIM23 | sc-514463 | Santa Cruz |
| Anti-phospho-DDR1 (pTyr513) | SAB4504671 | Sigma |
| anti-DDR1 | sc-390268 | Santa Cruz |
| anti-KEAP1 | 8047 | CST |
| anti-NF-κB p65 | 8242 | CST |
| anti-Histone H3 | A2348 | ABclonal |
| anti-CD326 (EpCAM) | 13-5791-80 | ThermoFisher |
| anti-IKKα | MA5-16157 | Invitrogen |
| anti-Actin | A4700 | Sigma |
| anti-GFP | A-11122 | ThermoFisher |
| anti-GFP/YFP/CFP | ab13970 | Abcam |
| anti-Flag | F3165, F7425 | Sigma |
| anti-TFAM | ab131607 | Abcam |
| anti-PGC1α | ABE868 | Sigma |
| anti-Phospho-AMPKα (Thr172) | 2535 | CST |
| anti-AMPKα | 5832 | CST |
| anti-6X His tag | ab18184 | Abcam |
| anti-E-Cadherin | 3195 | CST |
| anti-CD138/SDC1 | 36-2900 | ThermoFisher |
| anti-NHE-1 | sc-136239 | Santa Cruz |
| anti-PI3 Kinase p110γ | 5405 | CST |
| anti-ATP5A | sc-136178 | Santa Cruz |
| anti-ATP5B | MAB3494 | Sigma |
| anti-UQCRC2 | sc-390378 | Santa Cruz |
| anti-SDHB | sc-271548 | Santa Cruz |
| | 92649 | CST |
| anti-NDUFB7 | sc-365552 | Santa Cruz |
| anti-COX1/MT-CO1 | 62101 | CST |
| anti-αSMA | ab5694 | Abcam |
| anti-MMP1 | ab52631 | Abcam |
| anti-Ki67 | GTX16667 | GeneTex |
| anti-CDC42 | PA1-092 | ThermoFisher |
| anti-HSP90 | sc-13119 | Santa Cruz |
| anti-α-Amylase | A8273 | Sigma |
| anti-cytokeratin 19 | sc-33111 | Santa Cruz |
| anti-SOX9 | sc-20095 | Santa Cruz |
| anti-cytokeratin 18 | GTX105624 | GeneTex |
| anti-LAIR1 | H00003903-D01P | ThermoFisher |
| anti-Endo180/MRC2 | sc-271148 | Santa Cruz |
| anti-Integrin β1/ITGB1 | sc-374429 | Santa Cruz |
| anti-CD45 | 14-0451-85 | ThermoFisher |
| anti-CD68 | MA5-13324 | ThermoFisher |
| anti-CD163 | ab182422 | Abcam |
| anti-F4/80 | MF48000 | ThermoFisher |
| anti-CD4 | ab183685 | Abcam |
| anti-Ki67 | ab15580 | Abcam |
| anti-CD8 | ab217344 | Abcam |
| HRP goat anti-chicken IgY | sc-2428 | Santa Cruz |
| HRP goat anti-rabbit IgG | 7074 | CST |
| HRP horse anti-mouse IgG | 7076 | CST |
| HRP streptavidin | 554066 | Pharmingen |
| Biotin goat anti-mouse IgG | 553999 | Pharmingen |
| Biotin goat anti-rabbit IgG | 550338 | Pharmingen |
| Biotin mouse anti-goat IgG | sc-2489 | Santa Cruz |

Chromatin Immunoprecipitation

Cells were cross-linked with 1% formaldehyde for 10 min and the reaction was stopped with 0.125 M glycine for 5 min. The chromatin immunoprecipitation assay was performed as described¹¹. Cells were lysed and sonicated on ice to generate DNA fragments with an average length of 200-800 bp. After pre-clearing, 1% of each sample was saved as the input fraction. Immunoprecipitation was performed using antibodies that specifically recognize NRF2 (CST, 12721). DNA was eluted and purified from complexes, followed by PCR amplification of the target promoters or genomic loci using primers for mouse Tfam: 5′-GAGGCAGGGTCTCATG-3′ and 5′-CAAGCTGAGTTCTATC-3; 5′- TCTGGGCCATCTTGGG-3′ and 5′-CCATGGGCCTGGGCTG-3′.

Quantitative PCR Analysis

Total RNA and DNA were extracted using the All Prep DNA/RNA Mini Kit (Qiagen). RNA was reverse-transcribed using a Superscript VILO cDNA synthesis kit (Invitrogen). Quantitative (q)PCR was performed as described¹¹. Primers obtained from the NIH Primer-BLAST (https://www.ncbi.nlm.nih.gov/tools/primer-blast/index.cgi?LINK_LOC=BlastHome) are shown in Table 3.

TABLE 3
Real-time PCR Primer List

| Gene | Forward (5′-3′) | Reverse (5′-3′) |
|---|---|---|
| Pik3ca | GGACTGTGTGGGTCTCATCG | TCTCGCCCTTGTTCTIGTCC |
| Pik3cg | CTCTGGACCTGTGCCTTCTG | ATCTTTGAATGCCCCCGTGT |
| Cdc42 | GAGACTGCTGAAAAGCTGGCG | GGCTCTTCTTCGGTTCTGGA |
| | | GG |
| Nfe212 | AACAGAACGGCCCTAAAGCA | GGGATTCACGCATAGGAGCA |
| Nhe1 | TCATGAAGATAGGTTTCCAT | CGTCTGATTGCAGGAAGGGG |
| | GTGAT | |
| Sde1 | TCTGGCTCTGGCTCTGCG | GCCGTGACAAAGTATCTGGC |
| Sqstm1 | TGGGCAAGGAGGAGGCGACC | CCTCATCGCGGTAGTGCGCC |
| Egf | TTCTCACAAGGAAAGAGCAT | GTCCTGTCCCGTTAAGGAAA |
| | CTC | AC |
| m18s | AGCCCCTGCCCTTTGTACACA | CGATCCGAGGGCCTCACTA |

RNA-Seq Library Preparation, Processing and Analysis

Total RNA was isolated as described above from KPC samples grown on wild-type (n=3) or R/R (n=3) ECM as indicated. RNA purity was assessed by an Agilent 2100 Bioanalyzer. Five hundred nanograms of total RNA was enriched for poly-A-tailed RNA transcripts by double incubation with Oligo d(T) Magnetic Beads (NEB, S1419S) and fragmented for 9 min at 94° C. in 2× Superscript III first-strand buffer containing 10 mM DTT (Invitrogen, P2325). The reverse-transcription reaction was performed at 25° C. for 10 min followed by 50° C. for 50 min. The reverse-transcription product was purified with RNAClean XP (Beckman Coulter, A63987). Libraries were ligated with dual unique dual index (UDI) (IDT) or single UDI (Bioo Scientific), PCR-amplified for 11-13 cycles, size-selected using one-sided 0.8× AMPure clean-up beads, quantified using the Qubit dsDNA HS Assay Kit (Thermo Fisher Scientific) and sequenced on a HiSeq 4000 or NextSeq 500 (Illumina).

RNA-seq reads were aligned to the mouse genome (GRCm38/mm10) using STAR. Biological and technical replicates were used in all experiments. Quantification of transcripts was performed using HOMER (v.4.11). Principal component analysis (PCA) was obtained on the basis of transcripts per kilobase million (TPM) on all genes from all samples. Expression value for each transcript was calculated using the analyzeRepeats.pl tool of HOMER. Differential expression analysis was calculated using getDiffExpression.pl tool of HOMER. Pathway analyses were performed using the Molecular Signature Database of GSEA.

scRNA-seq Analysis

Samples from five primary tumours from patients with PDAC and one PDAC liver metastasis were obtained33 and analysed separately to better identify cell heterogeneity and clusters. The datasets were processed in R (v.4.0.2) and Seurat34 (v.4.0.5) and cells with at least 200 genes and genes expressed in at least 3 cells were retained for further quality control analysis for the percentage of mitochondrial genes expressed, total genes expressed and unique molecular identifier (UMI) counts. The gene-cell barcode matrix obtained after quality control analysis was log-normalized and 3,000 variable genes were identified and scaled to perform PCA. The five PDAC primary patient samples were then batch-corrected and integrated using a reciprocal PCA (RPCA) pipeline in Seurat using ‘FindIntegrationAnchors’ and ‘IntegrateData’ functions. The ‘integrated’ assay was again scaled to perform PCA. The top significant principal components of PCA were identified using ‘ElbowPlot’ in each dataset. To cluster and visualize the cells, ‘FindNeighbours’, ‘FindClusters’ and ‘RunUMAP’ functions were used on the top identified principal components in each dataset.

The cell types were identified by manual annotation of well-known makers³³, namely: epithelial-tumour cells (EPCAM and KRT8), pancreatic epithelial cells (CPA1 and CTRB 1), T cells (CD3D and IL7R), myeloid cells (CD14, CD68, FCGR3A and LYZ), NK cells (NKG7 and GNLY), B cells (CD79A and MS4A1), dendritic cells (FCGR1A and CPA3), endothelial cells (PECAM1, KDR and CDH5), fibroblasts (ACTA2, COL1A1, COLEC11 and DCN), vascular smooth muscle cells (MYH11 and ACTA2), hepatocytes (ALB, APOE and CPS1), cholangiocytes (ANXA4, KRT7 and SOX9), plasma cells (JCHAIN and IGKC) and cycling cells (TOP2A and MKI67).

M1/M2 macrophages were designated as described³⁵: M1-like macrophages (AZIN1, CD38, CXCL10, CXCL9, FPR2, IL18, IL1B, IRF5, NIFKBIZ, TLR4, TNF and CD80) and M2-like macrophages (ALOX5, ARG1, CHIL3, CD163, IL10, IL1ORA, IL1ORB, IRF4, KIF4, MRC1, MYC, SOCS2 and TGM2).

The mean expression score for the M1 and M2 signatures were computed for each macrophage subcluster using ‘AddModuleScore’ function and clusters with a higher M1 or M2 signature score were assigned M1-like or M2-like annotation, respectively.

Metabolite Extraction and Analysis

Cells grown on a 12-well plate coated with or without ECM. Metabolite extraction and analysis were performed as before¹¹. Gas chromatography-mass spectrometry (GC-MS) analysis was performed using an Agilent 6890 gas chromatograph equipped with a 30-m DB-35MS capillary column connected to an Agilent 5975B mass spectrometer operating under electron impact ionization at 70 eV. For measurement of amino acids, the gas chromatograph oven temperature was held at 100° C. for 3 min and increased to 300° C. at 3.5° C. per min. The mass spectrometer source and quadrupole were held at 23° C. and 150° C., respectively, and the detector was run in scanning mode, recording ion abundance in the range of 100-605 m/z. Mole per cent enrichments of stable isotopes in metabolite pools were determined by integrating the appropriate ion fragments and correcting for natural isotope abundance as previously described³⁶.

Cell Viability Assay

Cells were plated in 96-well plates coated with or without ECM at a density of 3,000 cells (MIA, 1305) or 1,500 cells (KPC or KC) per well and incubated overnight before treatment. 7rh (500 nM), ML120B (10 μM), EIPA (10.5 μM), IPI549 (600 nM), MBQ-167 (500 nM), MRT68921 (600 nM) or ML385 (10 μM), or their combinations, were added to the wells in the presence of complete medium (CM), LG medium or LQ medium for 72 h. Cell viability was determined with a Cell Counting Kit-8 assay (Glpbio). Optical density was read at 450 nm and analysed using a microplate reader with SoftMax 6.5 software (FilterMax F5, Molecular Devices). For all experiments, the medium was replaced every 24 h.

Luminescence ATP Detection Assay

KPC or KC cells were grown on 96-well plates coated with or without the indicated ECM in the presence of 100 μl CM or LG medium with or without EIPA (10.5 μM), MBQ-167 (500 nM), MRT68921 (600 nM)

or their combinations for 24 h. Then the cell number was measured. Intracellular ATP was determined with a luminescence ATP detection assay system (PerkinElmer) according to the manufacturer's protocol. Finally, luminescence was measured and normalized to cell number.

L-Amino Acid Assay

KPC or KC cells were grown on six-well plates coated with or without the indicated ECM in the presence of 100 μl LG medium with or without EIPA (10.5 μM), MRT68921 (600 nM) or their combinations for 24 h. Total amounts of free 1-amino acids (except for glycine) were measured using an L-Amino Acid Assay Kit (Colorimetric, antibodies) according to the manufacturer's protocol. The concentration of 1-amino acids was calculated within samples by comparing the sample optical density to the standard curve and normalized to cell number.

Statistics and Reproducibility

Macropinosomes or mitochondria were quantified by using the ‘Analyze Particles’ feature in Image J (NIH). Macropinocytotic uptake index³⁷ or mitochondria number was computed by the macropinosome or mitochondria area in relation to the total cell area for each field and then by determining the average across all the fields (six fields). Tumour area (%) was quantified by using the ‘Polygon’ and ‘Measure’ feature in Fiji Image J and was computed by tumour area in relation to total area for each field and then by determining the average across all the fields (five fields). Positive area of protein expression in tumour (%) was quantified by using ‘Colour Deconvolution’, ‘H DAB’, and ‘Analyze Particles’ feature in Fiji Image J and was computed by the protein-positive area in relation to the tumour area for each field and then by determining the average across all the fields (5-6 fields). These measurements were done on randomly selected fields of view. A two-tailed unpaired Student's t-test was performed for statistical analysis using GraphPad Prism software. Data are presented as mean±s.e.m. Kaplan—Meier survival curves were analysed by log-rank test. Statistical correlation between Col I—DDR1—NRF2 signaling proteins in human PDAC specimens was determined by two-tailed chi-squared test. (****P<0.0001, ***P<0.001, **P<0.01 and *P<0.05). All experiments except the IHC analysis of 106 human specimens were repeated at least 3 times.

Additional supporting information is provided in Su H. et al. (2022) Nature 610, 366-372, incorporated herein by reference.

Equivalents

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

1. Makohon-Moore, A. & Iacobuzio-Donahue, C. A. Pancreatic cancer biology and genetics from an evolutionary perspective. Nat. Rev. Cancer 16, 553-565 (2016).
2. Grzesiak, J. J. & Bouvet, M. The α2β1 integrin mediates the malignant phenotype on type I collagen in pancreatic cancer cell lines. Br. J. Cancer 94, 1311-1319 (2006).
3. Beatty, G. L. et al. CD40 agonists alter tumor stroma and show efficacy against pancreatic carcinoma in mice and humans. Science 331, 1612-1616 (2011).
4. Rhim, A. D. et al. Stromal elements act to restrain, rather than support, pancreatic ductal adenocarcinoma. Cancer Cell 25, 735-747 (2014).
5. Erkan, M. et al. The activated stroma index is a novel and independent prognostic marker in pancreatic ductal adenocarcinoma. Clin. Gastroenterol. Hepatol. 6, 1155-1161 (2008).
6. Armstrong, T. et al. Type I collagen promotes the malignant phenotype of pancreatic ductal adenocarcinoma. Clin. Cancer Res. 10, 7427-7437 (2004).
7. Ramanathan, R. K. et al. Phase MAI randomized study of FOLFIRINOX plus pegylated recombinant human hyaluronidase versus FOLFIRINOX alone in patients with metastatic pancreatic adenocarcinoma: SWOG 51313. J. Clin. Oncol. 37, 1062-1069 (2019).
8. Wu, H. et al. Generation of collagenase-resistant collagen by site-directed mutagenesis of murine pro alpha 1(I) collagen gene. Proc. Natl Acad. Sci. USA 87, 5888-5892 (1990).
9. Zhang, D. Y. & Friedman, S. L. Fibrosis-dependent mechanisms of hepatocarcinogenesis. Hepatol. 56, 769-775 (2012).
10. Baglieri, J. et al. Nondegradable collagen increases liver fibrosis but not hepatocellular carcinoma in mice. Am. J. Pathol. 191, 1564-1579 (2021).
11. Su, H. et al. Cancer cells escape autophagy inhibition via NRF2-induced macropinocytosis. Cancer Cell 39, 678-693 (2021).
12. Criscuolo, D., Avolio, R., Matassa, D. S. & Esposito, F. Targeting mitochondrial protein expression as a future approach for cancer therapy. Front. Oncol. 11, 797265 (2021).
13. Vogel, W., Gish, G. D., Alves, F. & Pawson, T. The discoidin domain receptor tyrosine kinases are activated by collagen. Mol. Cell 1, 13-23 (1997).
14. Das, S. et al. Discoidin domain receptor 1 receptor tyrosine kinase induces cyclooxygenase-2 and promotes chemoresistance through nuclear factor-kappaB pathway activation. Cancer Res. 66, 8123-8130 (2006).
15. Zhong, Z. et al. NF-κB restricts inflammasome activation via elimination of damaged mitochondria. Cell 164, 896-910 (2016).
16. Juskaite, V., Corcoran, D. S. & Leitinger, B. Collagen induces activation of DDR1 through lateral dimer association and phosphorylation between dimers. eLife 6, e25716 (2017).
17. Bhattacharjee, S. et al. Tumor restriction by type I collagen opposes tumor-promoting effects of cancer-associated fibroblasts. J. Clin. Invest. 131, 146987 (2021).
18. Yang, S. H. et al. Discoidin domain receptor 1 is associated with poor prognosis of non-small cell lung carcinomas. Oncol. Rep. 24, 311-319 (2010).
19. Turashvili, G. et al. Novel markers for differentiation of lobular and ductal invasive breast carcinomas by laser microdissection and microarray analysis. BMC Cancer 7, 55 (2007).
20. Sun, X. et al. Tumour DDR1 promotes collagen fibre alignment to instigate immune exclusion. Nature 599, 673-678 (2021).
21. Di Martino, J. S. et al. A tumor-derived type III collagen-rich ECM niche regulates tumor cell dormancy. Nat. Cancer 3, 90-107 (2022).
22. Picca, A. & Lezza, A. M. S. Regulation of mitochondrial biogenesis through TFAM-mitochondrial DNA interactions: useful insights from aging and calorie restriction studies. Mitochondrion 25, 67-75 (2015).
23. Scarpulla, R. C., Vega, R. B. & Kelly, D. P. Transcriptional integration of mitochondrial biogenesis. Trends Endocrinol. Metab. 23, 459-466 (2012).
24. Aguilera, K. Y. et al. Inhibition of discoidin domain receptor 1 reduces collagen-mediated tumorigenicity in pancreatic ductal adenocarcinoma. Mol. Cancer Ther. 16, 2473-2485 (2017).
25. Slapak, E. J., Duitman, J., Tekin, C., Bijlsma, M. F. & Spek, C. A. Matrix metalloproteases in pancreatic ductal adenocarcinoma: key drivers of disease progression? Biology 9, E80 (2020).
26. Aguilera, K. Y. et al. Collagen signaling enhances tumor progression after anti-VEGF therapy in a murine model of pancreatic ductal adenocarcinoma. Cancer Res. 74, 1032-1044 (2014).
27. Deng, J. et al. DDR1-induced neutrophil extracellular traps drive pancreatic cancer metastasis. JCI Insight 6, 146133 (2021).
28. Tones, M. P. et al. Novel pancreatic cancer cell lines derived from genetically engineered mouse models of spontaneous pancreatic adenocarcinoma: applications in diagnosis and therapy. PLoS ONE 8, e80580 (2013).
29. Zhang, T., Shen, S., Qu, J. & Ghaemmaghami, S. Global analysis of cellular protein flux quantifies the selectivity of basal autophagy. Cell Rep. 14, 2426-2439 (2016).
30. Su, H. et al. VPS34 acetylation controls its lipid kinase activity and the initiation of canonical and non-canonical autophagy. Mol. Cell 67, 907-921 (2017).
31. Liu, X. et al. A targeted mutation at the known collagenase cleavage site in mouse type I collagen impairs tissue remodeling. J. Cell Biol. 130, 227-237 (1995).
32. Monaghan, M. G., Kroll, S., Brucker, S. Y. & Schenke-Layland, K. Enabling multiphoton and second harmonic generation imaging in paraffin-embedded and histologically stained sections. Tissue Eng. Part C Methods 22, 517-523 (2016).
33. Lee, J. J. et al. Elucidation of tumor-stromal heterogeneity and the ligand—receptor interactome by single-cell transcriptomics in real-world pancreatic cancer biopsies. Clin. Cancer Res. 27, 5912-5921 (2021).
34. Hao, Y. et al. Integrated analysis of multimodal single-cell data. Cell 184, 3573-3587 (2021).
35. Li, S. et al. Metabolism drives macrophage heterogeneity in the tumor microenvironment. Cell Rep. 39, 110609 (2022).
36. Kumar, A., Mitchener, J., King, Z. A. & Metallo, C. M. Escher-Trace: a web application for pathway-based visualization of stable isotope tracing data. BMC Bioinformatics 21, 297 (2020).
37. Commisso, C., Flinn, R. J. & Bar-Sagi, D. Determining the macropinocytic index of cells through a quantitative image-based assay. Nat. Protoc. 9, 182-192 (2014).

What is claimed is:

1. A method for treating a subject suffering from a cancer selected from a desmoplastic cancer, a fibrolytic cancer or pancreatic ductal adenocarcinoma (PDAC) that has a tumor that has a higher level of cleaved type I collagen (cCol I) as compared to a subject not suffering from the cancer or having the cancer with a lower level of cCol I and/or more favorable outcome, comprising administering an effective amount of an aggressive anti-tumor therapy or an effective amount of a therapy that inhibits DDR1-stimulated NF-κB or mitochondrial biogenesis.

2. The method of claim 1, wherein the tumor further has a higher level of DDR1 and/or NRF2 as compared to a subject not suffering from the cancer or having the cancer with a lower level of DDRI and/or NRF2 and/or a more favorable outcome.

3. The method of claim 1, wherein the cancer is pancreatic ductal adenocarcinoma.

4. The method of claim 1, wherein the cancer is primary or metastatic.

5. The method of claim 1, wherein the cancer has metastasized to the liver.

6. The method of claim 1, wherein the level of cCol I is detected by a method comprising immunohistochemical detection.

7. The method of claim 2, wherein the level of DDR1 and/or NRF2 is detected by a method comprising immunohistochemical detection.

8. The method of claim 1, wherein the subject is a mammal.

9. The method of claim 8, wherein the mammal is a canine, feline, equine, murine, or a human patient.

10. The method of claim 1, further comprising tumor resection or radiation therapy.

11. The method of claim 1, wherein the treatment comprises one or more of: inhibiting metastatic potential of the cancer; reduction in tumor size; a reduction in tumor burden, longer progression free survival and longer overall survival of the subject.

12. A method for determining if a subject suffering from a cancer selected from a desmoplastic cancer, a fibrolytic cancer or pancreatic ductal adenocarcinoma (PDAC) is more or less likely to experience a longer survival comprising detecting the level of cleaved type I collagen (cCol I), in a tumor sample isolated from the subject, wherein a lower level of cCol I as compared to a subject not suffering from the cancer or having the cancer but having a higher level of cCol I indicates that the subject is more likely to experience longer survival and a higher level of cCol I as compared to a subject not suffering from the cancer or having a lower level of cCol indicates that the subject is more likely to experience shorter survival.

13. The method of claim 12, further comprising detecting the level of DDR1 and/or NRF2 in the sample, wherein a higher level of DDR1 and/or NRF2 in the sample indicates that the subject is less likely to experience longer survival and subjects with a lower levelof DDR1 and/or NRF2 are more likely to have longer survival.

14. The method of claim 12, wherein the cancer is pancreatic ductal adenocarcinoma.

15. The method of claim 12, wherein the cancer is primary or metastatic.

16. The method of claim 12, wherein the cancer has metastasized to the liver.

17. The method of claim 12, wherein the detecting of the level of cCol I comprises immunohistochemical detection.

18. The method of claim 13, wherein the detecting of the level of DDR1 and/or NRF2 comprises immunohistochemical detection.

19. The method of claim 12, further comprising administering to the subject having a lower level of any one, two or three of cCol I, DDR1, and NRF2, an aggressive anti-tumor therapy.

20. The method of claim 12, further comprising administering to the subject having a higher level of any one, two or three of cCol I, DDR1, and NRF2, an effective amount of a therapy that inhibits DDR1-stimulated NF-κB or mitochondrial biogenesis.
